(19) United States
(12) Patent Application Publication
(10) Pub. No.: US 2026/0098031 A1
(43) Pub. Date: Apr. 9, 2026

(54) MONOCYCLIC COMPOUND HAVING GLP-1 RECEPTOR AGONIST ACTIVITY

(71) Applicant: Shionogi & Co., Ltd., Osaka (JP)

(72) Inventor: Yuji NISHIURA, Osaka-shi Osaka (JP)

(73) Assignee: Shionogi & Co., Ltd., Osaka (JP)

(21) Appl. No.: 19/112,482

(22) PCT Filed: Sep. 21, 2023
(86) PCT No.: PCT/JP2023/034291

§ 371 Date: Mar. 17, 2025

(30) Foreign Application Priority Data
Sep. 22, 2022 (JP) 2022-150853

(51) Int. Cl. C07D 405/14; A61K 31/4439

(52) U.S. Cl. CPC C07D 405/14; A61K 31/4439

(57) ABSTRACT

The present invention relates to a compound represented by the following formula (I) or a pharmaceutically acceptable salt thereof.

A compound represented by formula (I):

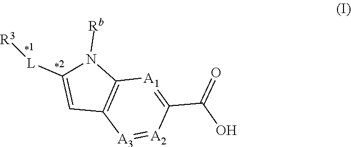

wherein
A₁ is C(R⁵) or N,
A₂ is C(R⁶) or N,
A₃ is C(R⁷) or N,
R⁵, R⁶, and R⁷ are each independently a hydrogen atom or the like,
R² is substituted or unsubstituted alkyl or the like,
-L- is a group represented by:

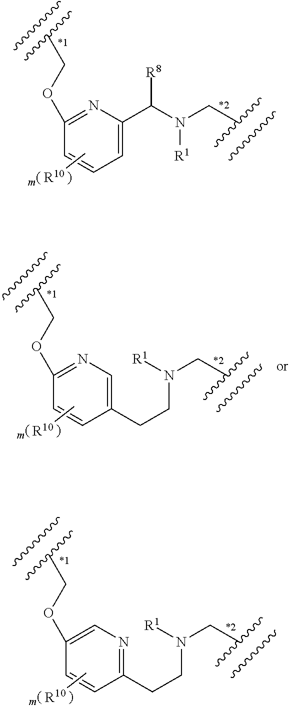

wherein
R¹ is a hydrogen atom or the like,
R⁸ is a hydrogen atom or the like,
R¹⁰s are each independently cyano or the like,
m is an integer of 1 to 3, and
R³ is phenyl optionally substituted with substituent group F, or the like,
the substituent group F: halogen, cyano, alkyl, haloalkyl, alkyloxy, and haloalkyloxy.

### TECHNICAL FIELD

The present invention relates to a compound that has GLP-1 receptor agonist activity and is useful as a therapeutic or prophylactic agent for diseases associated with the GLP-1 receptor, or its pharmaceutically acceptable salt, and a pharmaceutical composition containing thereof, particularly a prophylactic and/or therapeutic agent for non-insulin-dependent diabetes mellitus (type 2 diabetes mellitus) or obesity.

### BACKGROUND ART

Glucagon-like peptide-1 (GLP-1) is an incretin hormone that is secreted by L cells in the intestine in response to ingestion of food. GLP-1 is known to exhibit effects via the GLP-1 receptor such as promotion of glucose-dependent insulin secretion, reduction of glucagon secretion, delayed gastric emptying, and reduction of appetite. So far, it has been studied that an agonist of the GLP-1 receptor is used for treating diabetes and obesity (Non Patent Documents 1 and 2). Liraglutide, an analog formulation of human GLP-1, is known as a representative agonist, but it has been found that it exhibits a potent HbA1c lowering action and body weight reduction. Due to such attractive effects, a plurality of GLP-1 analog formulations have been put into practical use as therapeutic agents for diabetes and obesity. However, most of these GLP-1 analog formulations are sold as injectable formulations because of their poor oral absorbability. Therefore, development of an orally administrable GLP-1 receptor agonist is expected. Specifically, a method of causing semaglutide, which is an analog of GLP-1, to be orally absorbed by using an absorption promoting agent (Patent Document 1) has been put into practical use, but improvement of pharmaceutical properties such as bioavailability is required. In addition, attempts have been made to create a plurality of small molecule pharmaceutical products as non-peptidic GLP-1 receptor agonists (Patent Documents 2 to 58), but the substantially disclosed compounds have structures different from that of the compound of the present invention.

### PRIOR ART REFERENCES

### Patent Documents

[Patent Document 1] International Publication WO 2012/080471A
[Patent Document 2] International Publication WO 2009/111700A
[Patent Document 3] International Publication WO 2010/114824A
[Patent Document 4] International Publication WO 2018/056453A
[Patent Document 5] International Publication WO 2018/109607A
[Patent Document 6] International Publication WO 2019/239319A
[Patent Document 7] International Publication WO 2019/239371A
[Patent Document 8] International Publication WO 2020/103815A
[Patent Document 9] International Publication WO 2020/207474A
[Patent Document 10] International Publication WO 2020/263695A
[Patent Document 11] International Publication WO 2021/018023A
[Patent Document 12] International Publication WO 2021/081207A
[Patent Document 13] International Publication WO 2021/096284A
[Patent Document 14] International Publication WO 2021/096304A
[Patent Document 15] International Publication WO 2021/112538A
[Patent Document 16] International Publication WO 2021/155841A
[Patent Document 17] International Publication WO 2021/160127A
[Patent Document 18] International Publication WO 2021/187886A
[Patent Document 19] Chinese Patent Application Publication CN 113493447A
[Patent Document 20] International Publication WO 2021/197464A
[Patent Document 21] International Publication WO 2021/219019A
[Patent Document 22] Chinese Patent Application Publication CN 113480534A
[Patent Document 23] International Publication WO 2021/244645A
[Patent Document 24] International Publication WO 2021/249492A
[Patent Document 25] International Publication WO 2021/242817A
[Patent Document 26] Chinese Patent Application Publication CN 113773310A
[Patent Document 27] Chinese Patent Application Publication CN 113816948A
[Patent Document 28] Chinese Patent Application Publication CN 113801136A
[Patent Document 29] International Publication WO 2021/254470A
[Patent Document 30] International Publication WO 2021/259309A
[Patent Document 31] International Publication WO 2022/028572A
[Patent Document 32] International Publication WO 2022/031994A
[Patent Document 33] International Publication WO 2022/040600A
[Patent Document 34] International Publication WO 2022/042691A
[Patent Document 35] International Publication WO 2022/078380A
[Patent Document 36] International Publication WO 2022/078152A
[Patent Document 37] International Publication WO 2022/078407A
[Patent Document 38] International Publication WO 2022/109182A
[Patent Document 39] International Publication WO 2022/111624A
[Patent Document 40] International Publication WO 2022/116693A
[Patent Document 41] Chinese Patent Application Publication CN 114478497A
[Patent Document 42] Chinese Patent Application Publication CN 114716423A
[Patent Document 43] International Patent Application No. JP2022/13362
[Patent Document 44] International Publication WO 2022/068772A
[Patent Document 45] International Publication WO 2022/184849A
[Patent Document 46] International Publication WO 2022/192428A
[Patent Document 47] International Publication WO 2022/192430A
[Patent Document 48] International Publication WO 2022/199458A
[Patent Document 49] International Publication WO 2022/199661A
[Patent Document 50] International Publication WO 2022/219495A
[Patent Document 51] International Publication WO 2022/225914A
[Patent Document 52] International Publication WO 2022/225941A
[Patent Document 53] International Publication WO 2022/228490A
[Patent Document 54] International Publication WO 2022/246019A
[Patent Document 55] International Publication WO 2022/268152A
[Patent Document 56] Chinese Patent Application Publication CN 115594669A
[Patent Document 57]U.S. Patent Application Publication No. 20220396569
[Patent Document 58] International Publication WO 2023/138684A

### Non-Patent Documents

[Non-patent Document 1] Lancet 374, 1606-1616 (2009)
[Non-patent Document 2] Clin. Invest. 2, 59-72 (2012)

### SUMMARY OF THE INVENTION

### Problems to be Solved by the Invention

An object of the present invention is to provide a compound that has GLP-1 receptor agonist activity and is useful as a therapeutic or prophylactic agent for diseases relating to the GLP-1 receptor, or its pharmaceutically acceptable salt, and a pharmaceutical composition containing thereof, particularly a prophylactic and/or therapeutic agent for non-insulin-dependent diabetes mellitus (type 2 diabetes mellitus) or obesity.

### Means for Solving the Problem

The present invention relates to the following.
[1]A compound represented by formula (I):

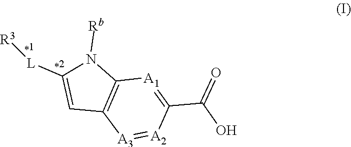

wherein
A₁ is C(R⁵) or N,
A₂ is C(R⁶) or N,
A₃ is C(R⁷) or N,
R⁵, R⁶, and R⁷ are each independently a hydrogen atom, halogen, cyano, substituted or unsubstituted alkyl, substituted or unsubstituted alkyloxy, substituted or unsubstituted aromatic heterocyclyl, or substituted or unsubstituted non-aromatic carbocyclyl,
R² is substituted or unsubstituted alkyl, or substituted or unsubstituted non-aromatic heterocyclyl,
-L- is a group represented by:

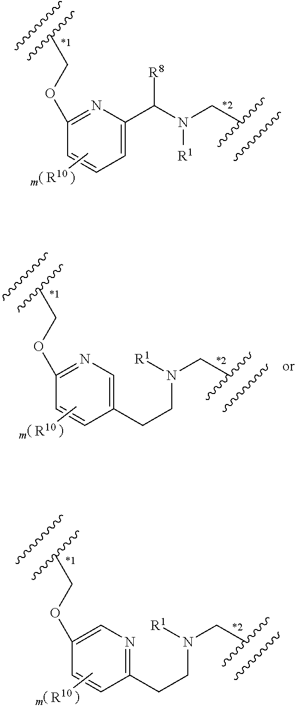

wherein
R¹ is a hydrogen atom, or substituted or unsubstituted alkyl,
R⁸ is a hydrogen atom, or substituted or unsubstituted alkyl,
R¹⁰s are each independently cyano, halogen, or substituted or unsubstituted alkyl, and
m is an integer of 1 to 3, and
R³ is phenyl optionally substituted with substituent group F, 5- or 6-membered aromatic heterocyclyl optionally substituted with substituent group F, bicyclic 9- or 10-membered aromatic heterocyclyl optionally substituted with substituent group F, or 5- to 12-membered non-aromatic heterocyclyl optionally substituted with substituent group F,
the substituent group F: halogen, cyano, alkyl, haloalkyl, alkyloxy, and haloalkyloxy,
or a pharmaceutically acceptable salt thereof.
[2] The compound according to the above [1] or a pharmaceutically acceptable salt thereof, wherein R² is alkyl substituted with substituted or unsubstituted non-aromatic heterocyclyl, or alkyl substituted with substituted or unsubstituted aromatic heterocyclyl.
[3] The compound according to the above [1] or [2] or a pharmaceutically acceptable salt thereof, wherein -L- is represented by:

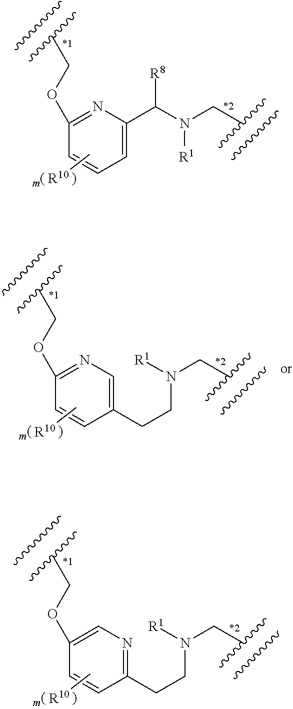

wherein R^{10a} and R^{10b} are each independently a hydrogen atom, cyano, halogen, or substituted or unsubstituted alkyl, and R¹ has the same meaning as in the above [1].
[4] The compound according to the above [3] or a pharmaceutically acceptable salt thereof, wherein R^{10b} is halogen, alkyl, or haloalkyl.
[5] The compound according to any one of the above [1] to [4] or a pharmaceutically acceptable salt thereof, wherein R³ is a group below:

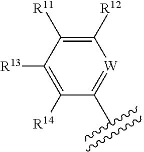

wherein
W is N, or CR¹⁵,
R¹¹ is a hydrogen atom, halogen, cyano, alkyl, haloalkyl, alkyloxy, or haloalkyloxy,
R¹² and R¹³ are each independently a hydrogen atom or halogen,
R¹⁴ and R¹⁵ are each independently a hydrogen atom, halogen, cyano, alkyl, haloalkyl, alkyloxy, or haloalkyloxy,
R¹¹ and R¹² may be taken together to form 5-membered aromatic heterocycle optionally substituted with substituent group F, or 5- to 7-membered non-aromatic heterocycle optionally substituted with substituent group F,
R¹¹ and R¹³ may be taken together to form 5-membered aromatic heterocycle optionally substituted with substituent group F, or 5- to 7-membered non-aromatic heterocycle optionally substituted with substituent group F, and
R¹³ and R¹⁴ may be taken together to form 5-membered aromatic heterocycle optionally substituted with substituent group F, or 5- to 7-membered non-aromatic heterocycle optionally substituted with substituent group F.
[6] The compound according to [5] or a pharmaceutically acceptable salt thereof, wherein R³ is any one of the following groups:

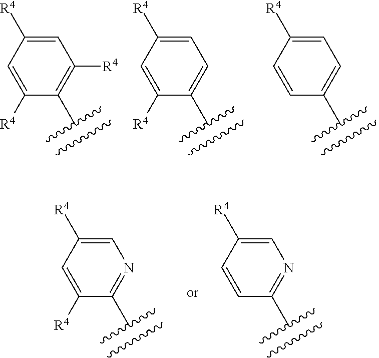

wherein R⁴s are each independently halogen, cyano, alkyl, haloalkyl, alkyloxy, or haloalkyloxy.
[7] The compound according to [6] or a pharmaceutically acceptable salt thereof, wherein R⁴s are each independently a fluorine atom, a chlorine atom, cyano, methyl, methyloxy, or difluoromethyloxy.
[8] The compound according to any one of the above [1] to [7] or a pharmaceutically acceptable salt thereof, wherein
   (i) A₁ is C(R⁵), A₂ is C(R⁶), and A₃ is C(R⁷),
   (ii) A₁ is N, A₂ is C(R⁶), and A₃ is C(R⁷),
   (iii) A₁ is C(R⁵), A₂ is C(R⁶), and A₃ is N, or
   (iv) A₁ is N, A₂ is C(R⁶), and A₃ is N.
[9] The compound according to the above [8] or a pharmaceutically acceptable salt thereof, wherein
   (i) A₁ is C(R⁵), A₂ is C(R⁶), and A₃ is C(R⁷), or
   (ii) A₁ is N, A₂ is C(R⁶), and A₃ is C(R⁷).
[10] The compound according to any one of the above [1] to [9] or a pharmaceutically acceptable salt thereof, wherein R⁵, R⁶, and R⁷ are each independently a hydrogen atom, halogen, alkyl, alkyloxy, or 5- or 6-membered aromatic heterocyclyl optionally substituted with a substituent group E, wherein the substituent group E is halogen, alkyl, haloalkyl, alkyloxy, haloalkyloxy.
[11] The compound according to any one of [1] to [10] or a pharmaceutically acceptable salt thereof, wherein R² is oxetanylmethyl.
[12]A pharmaceutical composition comprising the compound according to any one of the above [1] to [11] or a pharmaceutically acceptable salt thereof.
[13] The pharmaceutical composition according to [12], which is a GLP-1 receptor agonist.
[14]A method for treating and/or preventing a disease associated with GLP-1 receptor, comprising administering the compound according to any one of the above[1] to [11] or a pharmaceutically acceptable salt thereof.
[15] Use of the compound according to any one of the above [1] to [11] or a pharmaceutically acceptable salt thereof for manufacture of a medicament for treating and/or preventing a disease associated with GLP-1 receptor.
[16] The compound according to any one of the above [1] to [11] or a pharmaceutically acceptable salt thereof, for use in treating and/or preventing a disease associated with GLP-1 receptor.

### Effect of the Invention

The compound of the present invention has GLP-1 receptor agonist effect, and is useful as a prophylactic and/or therapeutic agent for diseases associated with GLP-1 receptor, particularly non-insulin-dependent diabetes mellitus, such as type 2 diabetes mellitus or obesity.

### MODE FOR CARRYING OUT THE INVENTION

Terms used in this description are explained below. Each term, unless otherwise indicated, has the same meaning when it is used alone or together with other terms.

The term “consisting of” means having only components.

The term “comprising” means not restricting to components and not excluding undescribed factors.

Hereinafter, the present invention will be described while showing exemplary embodiments. Throughout the present specification, it should be understood that, unless particularly stated otherwise, an expression of a singular form also includes the concept of a plural form thereof. Therefore, it should be understood that, unless particularly stated otherwise, an article for a singular form (for example, in the case of English, “a”, “an”, “the”, or the like) also includes the concept of a plural form thereof. Furthermore, it should be understood that, unless particularly stated otherwise, the terms used in the present specification are used in the meanings normally used in the above-described art. Accordingly, unless otherwise defined, all terminologies and scientific and technical terms used in the present specification have the same meanings as commonly understood by those having ordinary skill in the art to which the present invention belongs. In a case of contradiction, priority is given to the present specification including definitions.

“Halogen” includes a fluorine atom, a chlorine atom, a bromine atom and an iodine atom. Particularly, a fluorine atom and a chlorine atom are preferred.

“Alkyl” includes a C1 to C15, preferably C1 to C10, more preferably C1 to C6 and further preferably C1 to C4 linear or branched hydrocarbon group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl and n-decyl.

Preferred embodiments of “alkyl” include methyl, ethyl, n-propyl, isopropyl, n-butyl, isobutyl, sec-butyl, tert-butyl and n-pentyl. More preferred embodiments include methyl, ethyl, n-propyl, isopropyl and tert-butyl.

“Alkenyl” includes a C2 to C15, preferably a C2 to C10, more preferably a C2 to C6 and further preferably a C2 to C4 linear or branched hydrocarbon group having one or more double bond(s) at any position(s). Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl and pentadecenyl.

Preferred embodiments of “alkenyl” include vinyl, allyl, propenyl, isopropenyl and butenyl.

“Alkynyl” includes a C2 to C10, preferably a C2 to C8, more preferably a C2 to C6 and further preferably a C2 to C4 linear or branched hydrocarbon group having one or more triple bond(s) at any position(s). Alkynyl may further have double bond(s) at any position(s). Examples include ethynyl, propynyl, butynyl, pentynyl, hexynyl, heptynyl, octynyl, nonynyl and decynyl.

Preferred embodiments of “alkynyl” include ethynyl, propynyl, butynyl and pentynyl.

“Aromatic carbocyclyl” means a cyclic aromatic hydrocarbon group having a single ring or two or more rings. Examples include phenyl, naphthyl, anthryl and phenanthryl.

Preferred embodiments of the “aromatic carbocyclyl” include phenyl.

“Aromatic carbocycle” means a ring derived from the above “aromatic carbocyclyl”.

Preferred embodiment of “aromatic carbocycle” include a benzene ring.

“Non-aromatic carbocyclyl” means a cyclic saturated hydrocarbon group or a cyclic non-aromatic unsaturated hydrocarbon group, both having a single ring or two or more rings. A “non-aromatic carbocyclyl” having two or more rings also includes a fused ring group wherein a non-aromatic carbocyclyl having a single ring or two or more rings is fused with a ring of the above “aromatic carbocyclyl”, and the linking bond may be carried by any of the rings.

Examples include rings as follows.

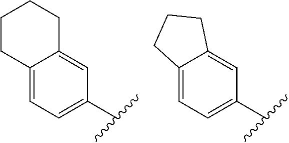 [SMILES: *c1ccc2c(c1)CCCC2 ; *c1ccc2c(c1)CCC2]

Furthermore, the “non-aromatic carbocyclyl” also includes a group having a bridged group or a group forming a spiro ring, such as follows.

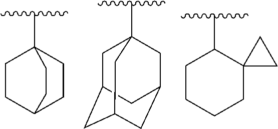

A non-aromatic carbocyclyl having a single ring is preferably C3 to C16, more preferably C3 to C12 and further preferably C4 to C8 non-aromatic carbocyclyl. Examples include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl and cyclohexadienyl.

The non-aromatic carbocyclyl which is polycyclic having two or more rings is preferably C8 to C20, more preferably C8 to C16 non-aromatic carbocyclyl. Examples include indanyl, indenyl, acenaphthyl, tetrahydronaphthyl and fluorenyl.

“Non-aromatic carbocycle” means a ring derived from the above “non-aromatic carbocyclyl”.

“Aromatic heterocyclyl” means an aromatic cyclyl having a single ring or two or more rings, which has one or more identical or different heteroatoms optionally selected from O, S, and N in the ring(s).

An aromatic heterocyclyl having two or more rings also includes a fused ring group wherein an aromatic heterocyclyl having a single ring or two or more rings is fused with a ring of the above “aromatic carbocyclyl”, and the linking bond may be carried by any of the rings.

The aromatic heterocyclyl having a single ring is preferably a 5- to 8-membered ring, and more preferably a 5-membered or 6-membered ring. Examples of 5-membered aromatic heterocyclyl include pyrrolyl, imidazolyl, pyrazolyl, triazolyl, tetrazolyl, furyl, thienyl, isoxazolyl, oxazolyl, oxadiazolyl, isothiazolyl, thiazolyl and thiadiazolyl. Examples of 6-membered aromatic heterocyclyl include pyridyl, pyridazinyl, pyrimidinyl, pyrazinyl and triazinyl. The aromatic heterocyclyl having two rings is preferably an 8- to 10-membered ring, and more preferably a 9-membered or 10-membered ring. Examples thereof include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl and thiazolopyridyl.

The aromatic heterocyclyl having three or more rings is preferably a 13- to 15-membered group. Examples include carbazolyl, acridinyl, xanthenyl, phenothiazinyl, phenoxathiinyl, phenoxazinyl and dibenzofuryl.

“Aromatic heterocycle” means a ring derived from the above “aromatic heterocyclyl”.

The aromatic heterocycle having a single ring is preferably a 5- to 8-membered ring and more preferably a 5-membered or 6-membered ring. Examples of the 5-membered aromatic heterocycle include a pyrroline ring, an imidazoline ring, a pyrazoline ring, a triazole ring, a tetrazole ring, a furan ring, a thiophen ring, an isoxazole ring, an oxazole ring, an oxadiazole ring, an isothiazole ring, a thiazole ring and a thiadiazole ring. Examples of 6-membered aromatic heterocycle include a pyridine ring, a pyridazine ring, a pyrimidine ring, a pyrazine ring and a triazine ring.

The aromatic heterocyclyl having two rings is preferably an 8- to 10-membered ring, and more preferably a 9-membered or 10-membered ring. Examples include an indole ring, an isoindole ring, an indazole ring, an indolizine ring, a quinoline ring, an isoquinoline ring, a cinnoline ring, a phthalazine ring, a quinazoline ring, a naphthyridine ring, a quinoxaline ring, a purine ring, a pteridine ring, a benzimidazole ring, a benzisoxazole ring, a benzoxazole ring, a benzoxadiazole ring, a benzisothiazole ring, a benzothiazole ring, a benzothiadiazole ring, a benzofuran ring, an isobenzofuran ring, a benzothiophene ring, a benzotriazole ring, an imidazopyridine ring, a triazolopyridine ring, an imidazothiazole ring, a pyrazinopyridazine ring, an oxazolopyridine ring and a thiazolopyridine ring.

An aromatic heterocycle having three or more rings is preferably a 13- to 15-membered group. Examples include a carbazole ring, an acridine ring, a xanthene ring, a phenothiazine ring, a phenoxathiin ring, a phenoxazine ring and a dibenzofuran ring.

“Non-aromatic heterocyclyl” means a non-aromatic cyclyl having a single ring or two or more rings, which has one or more identical or different heteroatoms optionally selected from O, S, and N in the ring(s). A non-aromatic heterocyclyl having two or more rings also includes a fused ring group wherein a non-aromatic heterocyclyl having a single ring or two or more rings is fused with a ring in each of the above “aromatic carbocyclyl”, “non-aromatic carbocyclyl” and/or “aromatic heterocyclyl”, as well as a non-aromatic carbocyclyl having a single ring or two or more rings is fused with a ring in the above “aromatic heterocyclyl”, and the linking bond may be carried by any of the rings.

Furthermore, the “non-aromatic heterocyclyl” also includes a group having a bridged group or a group forming a spiro ring, such as follows.

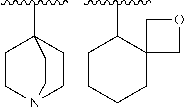

The non-aromatic heterocyclyl having a single ring is preferably a 3- to 8-membered and more preferably a 4- to 6-membered ring.

Examples of the 3-membered non-aromatic heterocyclyl include thiiranyl, oxiranyl and aziridinyl. Examples of 4-membered non-aromatic heterocyclyl include oxetanyl and azetidinyl. Examples of 5-membered non-aromatic heterocyclyl include oxathiolanyl, thiazolidinyl, pyrrolidinyl, pyrrolinyl, imidazolidinyl, imidazolinyl, pyrazolidinyl, pyrazolinyl, tetrahydrofuryl, dihydrothiazolyl, tetrahydroisothiazolyl, dioxolanyl, dioxolyl and thiolanyl. Examples of 6-membered non-aromatic heterocyclyl include dioxanyl, thianyl, piperidyl, piperazinyl, morpholinyl, morpholino, thiomorpholinyl, thiomorpholino, dihydropyridyl, tetrahydropyridyl, tetrahydropyranyl, dihydroxazinyl, tetrahydropyridazinyl, hexahydropyrimidinyl, dioxazinyl, thiinyl and thiazinyl. Examples of the 7-membered non-aromatic heterocyclyl include hexahydroazepinyl, tetrahydrodiazepinyl and oxepanyl.

The non-aromatic heterocyclyl having two or more rings is preferably an 8- to 20-membered group, and more preferably an 8- to 10-membered group. Examples include indolinyl, isoindolinyl, chromanyl and isochromanyl.

“Non-aromatic heterocycle” means a ring derived from the above “non-aromatic heterocyclyl”.

The alkyl moiety of “alkyloxy”, “haloalkyloxy”, “alkylcarbonyloxy”, “alkylcarbonyl”, “alkyloxycarbonyl”, “alkylsulfanyl”, “alkylsulfinyl”, “alkylsulfonyl”, “alkyloxyalkyloxy”, and “alkyloxyalkyl” have the same meanings as the aforementioned “alkyl”.

The alkenyl moiety of “alkenyloxy”, “alkenylcarbonyloxy”, “alkenylcarbonyl”, “alkenyloxycarbonyl”, “alkenylsulfanyl”, “alkenylsulfinyl”, and “alkenylsulfonyl” have the same meanings as the aforementioned“alkenyl”.

The alkynyl moiety of “alkynyloxy”, “alkynylcarbonyloxy”, “alkynylcarbonyl”, “alkynyloxycarbonyl”, “alkynylsulfanyl”, “alkynylsulfinyl”, and “alkynylsulfonyl” is synonymous with the aforementioned“alkynyl”.

In the present specification, the phrase “optionally substituted with substituent group A” means that “optionally substituted with one or more group(s) selected from substituent group A”. The same applies to substituent groups B, C, D, E, F, α, β, γ, γ′, and the like.

Substituents for “substituted alkyl”, “substituted alkenyl”, “substituted alkynyl”, “substituted alkyloxy”, “substituted alkenyloxy”, “substituted alkynyloxy”, “substituted alkylcarbonyloxy”, “substituted alkenylcarbonyloxy”, “substituted alkynylcarbonyloxy”, “substituted alkylcarbonyl”, “substituted alkenylcarbonyl”, “substituted alkynylcarbonyl”, “substituted alkyloxycarbonyl”, “substituted alkenyloxycarbonyl”, “substituted alkynyloxycarbonyl”, “substituted alkylsulfanyl”, “substituted alkenylsulfanyl”, “substituted alkynylsulfanyl”, “substituted alkylsulfinyl”, “substituted alkenylsulfinyl”, “substituted alkynylsulfinyl”, “substituted alkylsulfonyl”, “substituted alkenylsulfonyl”, “substituted alkynylsulfonyl” and the like include the following substituent group A. A carbon atom at any position may be bonded to one or more group(s) selected from the following substituent group A.

Substituent group A: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureido, amidino, guanidino, pentafluorothio, trialkylsilyl,
alkyloxy optionally substituted with substituent group α, alkenyloxy optionally substituted with substituent group α, alkynyloxy optionally substituted with substituent group α, alkylcarbonyloxy optionally substituted with substituent group α, alkenylcarbonyloxy optionally substituted with substituent group α, alkynylcarbonyloxy optionally substituted with substituent group α, alkylcarbonyl optionally substituted with substituent group α, alkenylcarbonyl optionally substituted with substituent group α, alkynylcarbonyl optionally substituted with substituent group α, alkyloxycarbonyl optionally substituted with substituent group α, alkenyloxycarbonyl optionally substituted with substituent group α, alkynyloxycarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
amino optionally substituted with substituent group β, imino optionally substituted with substituent group β, carbamoyl optionally substituted with substituent group β, sulfamoyl optionally substituted with substituent group β,
aromatic carbocyclyl optionally substituted with substituent group γ, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclyloxy optionally substituted with substituent group γ, non-aromatic carbocyclyloxy optionally substituted with substituent group γ′, aromatic heterocyclyloxy optionally substituted with substituent group γ, non-aromatic heterocyclyloxy optionally substituted with substituent group γ′, aromatic carbocyclylcarbonyloxy optionally substituted with substituent group γ, non-aromatic carbocyclylcarbonyloxy optionally substituted with substituent group γ′, aromatic heterocyclylcarbonyloxy optionally substituted with substituent group γ, non-aromatic heterocyclylcarbonyloxy optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylalkyloxy optionally substituted with substituent group γ, non-aromatic carbocyclylalkyloxy optionally substituted with substituent group γ′, aromatic heterocyclylalkyloxy optionally substituted with substituent group γ, non-aromatic heterocyclylalkyloxy optionally substituted with substituent group γ′, aromatic carbocyclylalkyloxycarbonyl optionally substituted with substituent group γ, non-aromatic carbocyclylalkyloxycarbonyl optionally substituted with substituent group γ′, aromatic heterocyclylalkyloxycarbonyl optionally substituted with substituent group γ, non-aromatic heterocyclylalkyloxycarbonyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

Substituent group α: halogen, hydroxy, carboxy, alkyloxy, haloalkyloxy, alkenyloxy, alkynyloxy, sulfanyl and cyano.

Substituent group β: halogen, hydroxy, carboxy, cyano, alkyl optionally substituted with substituent group α, alkenyl optionally substituted with substituent group α, alkynyl optionally substituted with substituent group α, alkylcarbonyl optionally substituted with substituent group α, alkenylcarbonyl optionally substituted with substituent group α, alkynylcarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
aromatic carbocyclyl optionally substituted with substituent group γ, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclylalkyl optionally substituted with substituent group γ, non-aromatic carbocyclylalkyl optionally substituted with substituent group γ′, aromatic heterocyclylalkyl optionally substituted with substituent group γ, non-aromatic heterocyclylalkyl optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

Substituent group γ: substituent group α, alkyl, haloalkyl, cyanoalkyl, hydroxyalkyl, alkenyl, alkynyl, alkylcarbonyl, haloalkylcarbonyl, alkenylcarbonyl, and alkynylcarbonyl.

Substituent group γ′: substituent group γ and oxo.

The substituents on the rings of “aromatic carbocycle” and “aromatic heterocycle”, such as “substituted aromatic carbocyclyl”, “substituted aromatic heterocyclyl”, “substituted aromatic carbocyclyloxy”, “substituted aromatic heterocyclyloxy”, “substituted aromatic carbocyclylcarbonyloxy”, “substituted aromatic heterocyclylcarbonyloxy”, “substituted aromatic carbocyclylcarbonyl”, “substituted aromatic heterocyclylcarbonyl”, “substituted aromatic carbocyclyloxycarbonyl”, “substituted aromatic heterocyclyloxycarbonyl”, “substituted aromatic carbocyclylsulfanyl”, “substituted aromatic heterocyclylsulfanyl”, “substituted aromatic carbocyclylsulfinyl”, “substituted aromatic heterocyclylsulfinyl”, “substituted aromatic carbocyclylsulfonyl”, and “substituted aromatic heterocyclylsulfonyl” include the following substituent group B. An atom at any position on the ring may be bonded to one or more group(s) selected from the following substituent group B.

Substituent group B: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureido, amidino, guanidino, pentafluorothio, trialkylsilyl,
alkyl optionally substituted with substituent group α, alkenyl optionally substituted with substituent group α, alkynyl optionally substituted with substituent group α, alkyloxy optionally substituted with substituent group α, alkenyloxy optionally substituted with substituent group α, alkynyloxy optionally substituted with substituent group α, alkylcarbonyloxy optionally substituted with substituent group α, alkenylcarbonyloxy optionally substituted with substituent group α, alkynylcarbonyloxy optionally substituted with substituent group α, alkylcarbonyl optionally substituted with substituent group α, alkenylcarbonyl optionally substituted with substituent group α, alkynylcarbonyl optionally substituted with substituent group α, alkyloxycarbonyl optionally substituted with substituent group α, alkenyloxycarbonyl optionally substituted with substituent group α, alkynyloxycarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
amino optionally substituted with substituent group β, imino optionally substituted with substituent group β, carbamoyl optionally substituted with substituent group β, sulfamoyl optionally substituted with substituent group β,
aromatic carbocyclyl optionally substituted with substituent group γ, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclyloxy optionally substituted with substituent group γ, non-aromatic carbocyclyloxy optionally substituted with substituent group γ′, aromatic heterocyclyloxy optionally substituted with substituent group γ, non-aromatic heterocyclyloxy optionally substituted with substituent group γ′, aromatic carbocyclylcarbonyloxy optionally substituted with substituent group γ, non-aromatic carbocyclylcarbonyloxy optionally substituted with substituent group γ′, aromatic heterocyclylcarbonyloxy optionally substituted with substituent group γ, and non-aromatic heterocyclylcarbonyloxy optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylalkyl optionally substituted with substituent group γ, non-aromatic carbocyclylalkyl optionally substituted with substituent group γ′, aromatic heterocyclylalkyl optionally substituted with substituent group γ, non-aromatic heterocyclylalkyl optionally substituted with substituent group γ′, aromatic carbocyclylalkyloxy optionally substituted with substituent group γ, non-aromatic carbocyclylalkyloxy optionally substituted with substituent group γ′, aromatic heterocyclylalkyloxy optionally substituted with substituent group γ, non-aromatic heterocyclylalkyloxy optionally substituted with substituent group γ′, aromatic carbocyclylalkyloxycarbonyl optionally substituted with substituent group γ, non-aromatic carbocyclylalkyloxycarbonyl optionally substituted with substituent group γ′, aromatic heterocyclylalkyloxycarbonyl optionally substituted with substituent group γ, non-aromatic heterocyclylalkyloxycarbonyl optionally substituted with substituent group γ′, aromatic carbocyclylalkyloxyalkyl optionally substituted with substituent group γ, non-aromatic carbocyclylalkyloxyalkyl optionally substituted with substituent group γ′, aromatic heterocyclylalkyloxyalkyl optionally substituted with substituent group γ, non-aromatic heterocyclylalkyloxyalkyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

Cyclic substituents for “non-aromatic carbocycle” and “aromatic heterocycle” of “substituted non-aromatic carbocyclyl”, “substituted non-aromatic heterocyclyl”, “substituted non-aromatic carbocyclyloxy”, “substituted non-aromatic heterocyclyloxy”, “substituted non-aromatic carbocyclylcarbonyloxy”, “substituted non-aromatic heterocyclylcarbonyloxy”, “substituted non-aromatic carbocyclylcarbonyl”, “substituted non-aromatic heterocyclylcarbonyl”, “substituted non-aromatic carbocyclyloxycarbonyl”, “substituted non-aromatic heterocyclyloxycarbonyl”, “substituted non-aromatic carbocyclylsulfanyl”, “substituted non-aromatic heterocyclylsulfanyl”, “substituted non-aromatic carbocyclylsulfinyl”, “substituted non-aromatic heterocyclylsulfinyl”, “substituted non-aromatic carbocyclylsulfonyl” and “substituted non-aromatic heterocyclylsulfonyl” include the following substituent group C. An atom at an arbitrary position on the ring may be bonded to one or more groups selected from the following substituent group C.

Substituent group C: substituent group B and oxo.

When the “non-aromatic carbocycle”, the “non-aromatic heterocycle”, the “non-aromatic carbocyclyl” and the “non-aromatic heterocyclyl” are substituted with “oxo”, this means a ring in which two hydrogen atoms on a carbon atom are substituted as follows.

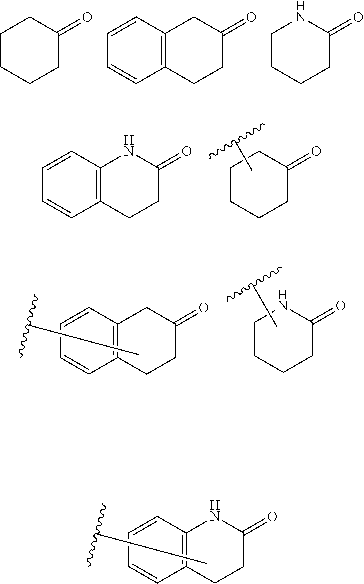

Examples of the substituents for “substituted amino”, “substituted imino”, “substituted carbamoyl” and “substituted sulfamoyl” include the following substituent group D. These moieties may be substituted with one or two group(s) selected from substituent group D.

Substituent group D: halogen, hydroxy, carboxy, cyano, alkyl optionally substituted with substituent group α, alkenyl optionally substituted with substituent group α, alkynyl optionally substituted with substituent group α, alkylcarbonyl optionally substituted with substituent group α, alkenylcarbonyl optionally substituted with substituent group α, alkynylcarbonyl optionally substituted with substituent group α, alkylsulfanyl optionally substituted with substituent group α, alkenylsulfanyl optionally substituted with substituent group α, alkynylsulfanyl optionally substituted with substituent group α, alkylsulfinyl optionally substituted with substituent group α, alkenylsulfinyl optionally substituted with substituent group α, alkynylsulfinyl optionally substituted with substituent group α, alkylsulfonyl optionally substituted with substituent group α, alkenylsulfonyl optionally substituted with substituent group α, alkynylsulfonyl optionally substituted with substituent group α,
amino optionally substituted with substituent group β, imino optionally substituted with substituent group β, carbamoyl optionally substituted with substituent group β, sulfamoyl optionally substituted with substituent group β,
aromatic carbocyclyl optionally substituted with substituent group γ, non-aromatic carbocyclyl optionally substituted with substituent group γ′, aromatic heterocyclyl optionally substituted with substituent group γ, non-aromatic heterocyclyl optionally substituted with substituent group γ′, aromatic carbocyclylalkyl optionally substituted with substituent group γ, non-aromatic carbocyclylalkyl optionally substituted with substituent group γ′, aromatic heterocyclylalkyl optionally substituted with substituent group γ, non-aromatic heterocyclylalkyl optionally substituted with substituent group γ′, aromatic carbocyclylcarbonyl optionally substituted with substituent group γ, non-aromatic carbocyclylcarbonyl optionally substituted with substituent group γ′, aromatic heterocyclylcarbonyl optionally substituted with substituent group γ, non-aromatic heterocyclylcarbonyl optionally substituted with substituent group γ′, aromatic carbocyclyloxycarbonyl optionally substituted with substituent group γ, non-aromatic carbocyclyloxycarbonyl optionally substituted with substituent group γ′, aromatic heterocyclyloxycarbonyl optionally substituted with substituent group γ, non-aromatic heterocyclyloxycarbonyl optionally substituted with substituent group γ′, aromatic carbocyclylsulfanyl optionally substituted with substituent group γ, non-aromatic carbocyclylsulfanyl optionally substituted with substituent group γ′, aromatic heterocyclylsulfanyl optionally substituted with substituent group γ, non-aromatic heterocyclylsulfanyl optionally substituted with substituent group γ′, aromatic carbocyclylsulfinyl optionally substituted with substituent group γ, non-aromatic carbocyclylsulfinyl optionally substituted with substituent group γ′, aromatic heterocyclylsulfinyl optionally substituted with substituent group γ, non-aromatic heterocyclylsulfinyl optionally substituted with substituent group γ′, aromatic carbocyclylsulfonyl optionally substituted with substituent group γ, non-aromatic carbocyclylsulfonyl optionally substituted with substituent group γ′, aromatic heterocyclylsulfonyl optionally substituted with substituent group γ, and non-aromatic heterocyclylsulfonyl optionally substituted with substituent group γ′.

In formula (I), the bond attached with *1 of -L- is bonded to R³, and the bond attached with *2 of -L- is bonded to an imidazole ring.

A preferred embodiment of each symbol in the compound represented by formula (I) is described below. Examples of the compound represented by formula (I) include embodiments of all combinations of specific examples described below.
A₁ is C(R⁵) or N.
A₂ is C(R⁶) or N.
As is C(R⁷) or N.
A₁, A₂, and A₃ are preferably
(i) A₁ is C(R⁵), A₂ is C(R⁶), and A₃ is C(R⁷),
(ii) A₁ is N, A₂ is C(R⁶), and A₃ is C(R⁷),
(iii) A₁ is C(R⁵), A₂ is C(R⁶), and A₃ is N, or
(iv) A₁ is N, A₂ is C(R⁶), and A₃ is N.
A₁, A₂, and A₃ are more preferably
(i) A₁ is C(R⁵), A₂ is C(R⁶), and A₃ is C(R⁷), or
(ii) A₁ is N, A₂ is C(R⁵), and A₃ is C(R⁷).
R⁵, R⁶, and R⁷ are each independently a hydrogen atom, halogen, cyano, substituted or unsubstituted alkyl (wherein the substituent is, for example, halogen), substituted or unsubstituted alkyloxy (wherein the substituent is, for example, halogen), substituted or unsubstituted aromatic heterocyclyl (wherein the substituent is, for example, halogen, alkyl, haloalkyl, alkyloxy, haloalkyloxy), or substituted or unsubstituted non-aromatic carbocyclyl (wherein the substituent is, for example, halogen, alkyl, haloalkyl, alkyloxy, haloalkyloxy).
R⁵, R⁶, and R⁷ are preferably each independently a hydrogen atom, halogen, alkyl, alkyloxy, or 5- or 6-membered aromatic heterocyclyl optionally substituted with substituent group E.

Substituent group E: halogen, alkyl, haloalkyl, alkyloxy, haloalkyloxy.

The substituent group E is preferably a group selected from halogen and alkyl.
R⁵ is preferably a hydrogen atom or halogen, and more preferably a hydrogen atom or a fluorine atom.
R⁶ is preferably a hydrogen atom.
R⁷ is preferably a hydrogen atom, halogen, alkyloxy, or methylpyrazolyl, and more preferably a hydrogen atom, a fluorine atom, methyloxy, or methylpyrazolyl.
R² is substituted or unsubstituted alkyl, or substituted or unsubstituted non-aromatic heterocyclyl.
R² is preferably alkyl, alkyl substituted with non-aromatic heterocyclyl, or alkyl substituted with aromatic heterocyclyl, and more preferably oxetanylalkyl or alkylimidazolylalkyl.
R² is most preferably oxetanylmethyl.
-L- is any one of groups below:

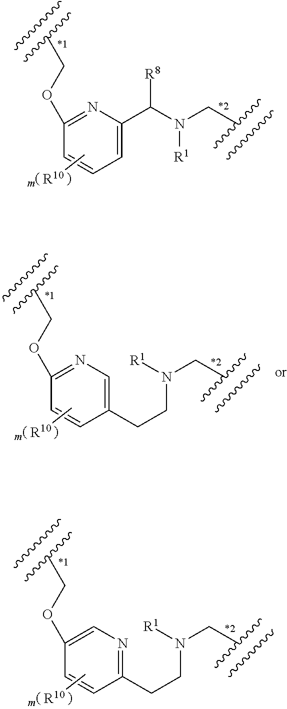

-L- is preferably any one of the following groups:

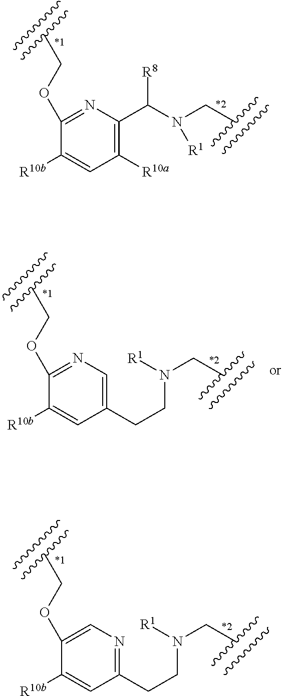

-L- is more preferably the following:

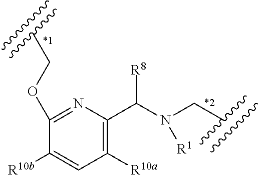

R¹ is a hydrogen atom, or substituted or unsubstituted alkyl (wherein the substituent is, for example, halogen), and preferably alkyl.
R⁸ is a hydrogen atom, or substituted or unsubstituted alkyl (wherein the substituent is, for example, halogen), and preferably alkyl.
R¹⁰s are each independently cyano, halogen, or substituted or unsubstituted alkyl (wherein the substituent is, for example, halogen), and preferably cyano, fluorine atom, chlorine atom, methyl, difluoromethyl, or trifluoromethyl.
R^{10a} is a hydrogen atom, cyano, halogen, or substituted or unsubstituted alkyl (wherein the substituent is, for example, halogen), and preferably cyano, fluorine atom, chlorine atom, methyl, difluoromethyl, or trifluoromethyl.
R^{10b} is a hydrogen atom, cyano, halogen, or substituted or unsubstituted alkyl (wherein the substituent is, for example, halogen), and preferably cyano, fluorine atom, chlorine atom, methyl, difluoromethyl, or trifluoromethyl, and more preferably trifluoromethyl.
R³ is phenyl optionally substituted with substituent group F, 5- or 6-membered aromatic heterocyclyl optionally substituted with substituent group F, bicyclic 9- or 10-membered aromatic heterocyclyl optionally substituted with substituent group F, or 5- to 12-membered non-aromatic heterocyclyl optionally substituted with substituent group F.

Substituent group F: halogen, cyano, alkyl, haloalkyl, alkyloxy, and haloalkyloxy.
R³ is preferably the following group:

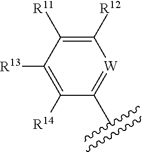

wherein
W is N, or CR¹⁵,
R¹¹ is a hydrogen atom, halogen, cyano, alkyl, haloalkyl, alkyloxy, or haloalkyloxy,
R¹² and R¹³ are each independently a hydrogen atom or halogen,
R¹⁴ and R¹⁵ are each independently a hydrogen atom, halogen, cyano, alkyl, haloalkyl, alkyloxy, or haloalkyloxy,
R¹¹ and R¹² may be taken together to form 5-membered aromatic heterocycle optionally substituted with substituent group F, or 5- to 7-membered non-aromatic heterocycle optionally substituted with substituent group F,
R¹¹ and R¹³ may be taken together to form 5-membered aromatic heterocycle optionally substituted with substituent group F, or 5- to 7-membered non-aromatic heterocycle optionally substituted with substituent group F, and
R¹³ and R¹⁴ may be taken together to form 5-membered aromatic heterocycle optionally substituted with substituent group F, or 5- to 7-membered non-aromatic heterocycle optionally substituted with substituent group F.
R³ is more preferably any one of the following groups:

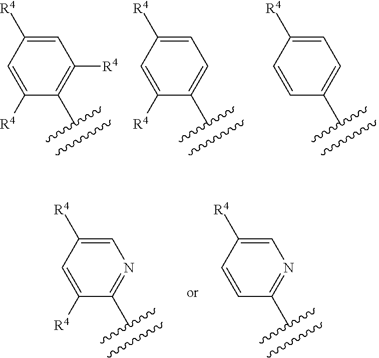

wherein R⁴s are each independently halogen, cyano, alkyl, haloalkyl, alkyloxy, or haloalkyloxy.

R⁴s are preferably each independently a fluorine atom, a chlorine atom, cyano, methyl, methyloxy, or difluoromethyloxy.

The compounds represented by formula (I) are not limited to specific isomers, but include all possible isomers (e.g., keto-enol isomers, imine-enamine isomers, diastereoisomers, optical isomers, rotational isomers, tautomers as shown below, etc.), racemates, or mixtures thereof.

One or more hydrogen, carbon and/or other atoms in the compounds represented by formula (I) may be replaced with isotopes of hydrogen, carbon and/or other atoms respectively. Examples of such isotopes include hydrogen, carbon, nitrogen, oxygen, phosphorus, sulfur, fluorine, iodine, and chlorine, as in the cases of ²H, ³H, ¹¹C, ¹³C, ¹⁴C, ¹⁵N, ¹⁸O, ¹⁷O, ³¹P, ³²P ³⁵S, ¹⁸F, ¹²³I, and ³⁶Cl, respectively. The compound represented by Formula (I) also includes a compound substituted with such an isotope. The compound substituted with the isotope is also useful as a pharmaceutical product and includes all radiolabeled forms of the compound represented by Formula (I). Furthermore, a “radiolabeling method” for producing the “radiolabeled forms” is also included in the present invention, and the “radiolabeled forms” are useful as tools for metabolic pharmacokinetics studies, studies on binding assay, and/or diagnostics.

A radiolabeled compound of the compounds represented by formula (I) can be prepared using well-known methods in the art. For example, a tritium-labeled compound represented by Formula (I) can be prepared by introducing tritium into a specific compound represented by Formula (I) by catalytic dehalogenation reaction using tritium. This method includes reacting a suitably halogen-substituted precursor of the compound represented by Formula (I) with tritium gas in the presence of a suitable catalyst such as Pd/C, in the presence or absence of a base. Regarding other appropriate methods for preparing tritium-labeled compounds, “Isotopes in the Physical and Biomedical Sciences, Vol. 1, Labeled Compounds (Part A), Chapter 6 (1987)” can be referred to. A ¹⁴C-labeled compound can be prepared by using a raw material having ¹⁴C carbon.

The pharmaceutically acceptable salts of the compounds represented by formula (I) include, for example, salts of the compounds represented by formula (I) with alkaline metal (e.g., lithium, sodium, potassium or the like), alkaline earth metal (e.g., calcium, barium or the like), magnesium, transition metal (e.g., zinc, iron or the like), ammonia, organic bases (e.g., trimethylamine, triethylamine, dicyclohexylamine, ethanolamine, diethanolamine, triethanolamine, meglumine, ethylenediamine, pyridine, picoline, quinoline or the like) or amino acids, or salts with inorganic acids (e.g., hydrochloric acid, sulfuric acid, nitric acid, carbonic acid, hydrobromic acid, phosphoric acid, hydroiodic acid or the like) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid or the like). Particularly, examples of salts include hydrochloric acid, phosphoric acid, tartaric acid, or methanesulfonic acid. These salts can be formed according to methods that are conventionally carried out.

The compounds represented by formula (I) or pharmaceutically acceptable salts thereof may form a solvate (e.g., a hydrate), a cocrystal and/or a crystal polymorph. The present invention also encompasses such various solvates, cocrystals and crystal polymorphs. The “solvate” may be one wherein any number of solvent molecules (e.g., water molecules or the like) is coordinated with the compounds represented by formula (I). When the compound represented by Formula (I) or a pharmaceutically acceptable salt thereof is allowed to stand in the atmosphere, the compound may absorb water, resulting in attachment of adsorbed water or formation of a hydrate. Recrystallization of the compound represented by Formula (I) or a pharmaceutically acceptable salt thereof may form a crystal polymorph. The term “cocrystal” means that the compound represented by Formula (I) or a salt and a counter molecule are present in the same crystal lattice, and may contain any number of counter molecules.

The compounds represented by formula (I) or pharmaceutically acceptable salts thereof may form prodrugs. The present invention also encompasses such various prodrugs. A prodrug is a derivative of a compound of the present invention having a group that can be chemically or metabolically degraded, and is a compound which becomes a pharmaceutically active compound of the present invention in vivo as a result of solvolysis or under physiological conditions. The prodrug includes, for example, a compound that is converted to the compound represented by Formula (I) through enzymatic oxidation, reduction, hydrolysis, or the like under physiological conditions in vivo, and a compound that is converted to the compound represented by Formula (I) through hydrolysis by gastric juice or the like. Methods for selecting and preparing suitable prodrug derivatives are described in, for example, “Design of Prodrugs, Elsevier, Amsterdam, 1985”. A prodrug may have activity per se.

When the compounds represented by formula (I) or pharmaceutically acceptable salts thereof have hydroxy group(s), prodrugs include acyloxy derivatives and sulfonyloxy derivatives that are prepared by, for example, reacting compounds having hydroxy group(s) with suitable acyl halide, suitable acid anhydride, suitable sulfonyl chloride, suitable sulfonyl anhydride and mixed anhydride, or with a condensing agent. Examples include CH₃COO—, C₂H₅COO—, tert-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃O-PhSO₃—, PhSO₃—, and p-CH₃PhSO₃—.

### (Method for Producing Compound of Present Invention)

The compound represented by Formula (I) can be produced, for example, by a general synthesis method described below. Starting materials and reaction reagents used in such synthesis are commercially available or can be synthesized according to methods well known in the art using compounds commercially available. Regarding extraction, purification, and the like, the treatments carried out in ordinary experiments of organic chemistry may be carried out.

The compounds of the present invention can be synthesized with reference to methods known in the art.

In the following steps, when a substituent which interferes with the reaction, e.g. hydroxy, mercapto, amino, formyl, carbonyl, carboxy, is possessed, the substituent is protected by the method such as those described in Protective Groups in Organic Synthesis, Theodora W Greene(John Wiley & Sons) in advance, and the protective group may be removed at a desirable step.

In addition, in the all steps below, an order of steps to be implemented may be appropriately changed, and each intermediate may be isolated, and used in a next step. All of reaction time, reaction temperature, solvents, reagents, protecting groups, etc. are mere exemplification and not limited as long as they do not cause an adverse effect on a reaction.

General procedures for the synthesis of the compounds of the present invention are described below. Starting materials and reaction reagents used in such synthesis are commercially available or can be synthesized according to methods well known in the art using compounds commercially available.

For example, the compounds represented by formula (I) of the present invention can be prepared by the general synthetic methods described below.

### General Synthetic Method 1

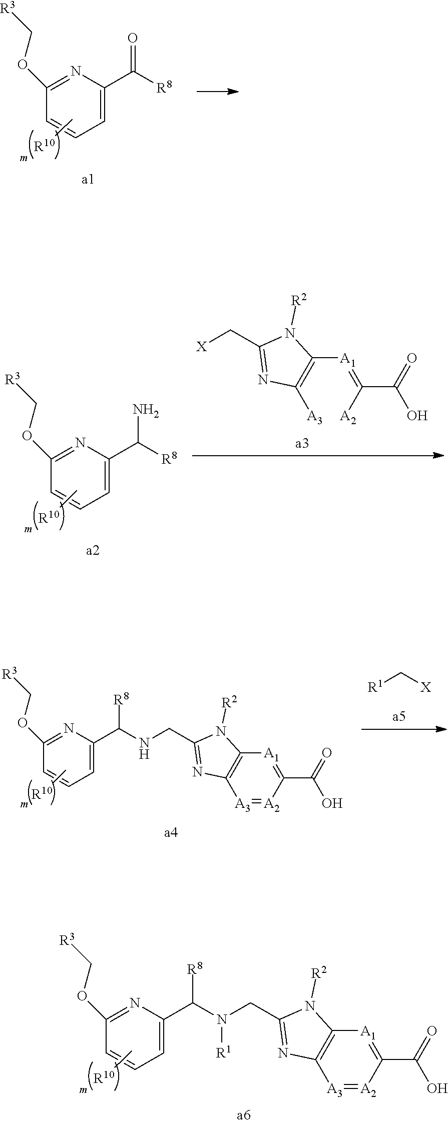

wherein X is a leaving group such as halogen, and other symbols have the same meaning as described above.

### Step 1

Compound a2 can be obtained by allowing an ammonium source and a reducing agent to act on Compound a1.

The reaction temperature is −20° C. to 70° C., and preferably 0° C. to 50° C.

The reaction time is 0.5 hours to 168 hours, and preferably 3 hours to 48 hours.

Examples of the ammonium source include ammonium formate, ammonium chloride, and ammonium acetate, and can be used at 1 to 20 molar equivalents relative to Compound a1.

Examples of the reducing agent include sodium borohydride, sodium cyanoborohydride, sodium triacetoxyborohydride, and 2-picolinborane, and the reducing agent can be used at 1 to 10 molar equivalents relative to Compound a1.

Examples of the reaction solvent include methanol, ethanol, tetrahydrofuran, acetonitrile, and dichloromethane, and one of or a mixture of these can be used.

### Step 2

Compound a4 can be obtained by allowing Compound a3 to act on Compound a2 in the presence of a base.

The reaction temperature is 0° C. to the reflux temperature of the solvent.

The reaction time is 0.5 hours to 12 hours, and preferably 1 hour to 6 hours.

Compound a3 can be used at 1 to 3 molar equivalents relative to Compound a2.

Examples of the base include potassium tert-butoxide, sodium tert-butoxide, sodium carbonate, potassium carbonate, and cesium carbonate, and the base can be used at 0.9 to 5 molar equivalents relative to Compound a2.

Examples of the reaction solvent include methanol, ethanol, acetonitrile, tetrahydrofuran, and dimethylformamide, and one of or a mixture of these can be used.

### Step 3

Compound a6 can be obtained by allowing Compound a5 to act on Compound a4 in the presence of a base.

The reaction temperature is 0° C. to the reflux temperature of the solvent.

The reaction time is 0.5 hours to 12 hours, and preferably 1 hour to 6 hours.

Compound a5 can be used at 1 to 20 molar equivalent(s) relative to Compound a4.

Examples of the base include potassium tert-butoxide, sodium tert-butoxide, sodium carbonate, potassium carbonate, and cesium carbonate, and the base can be used at 1 to 20 molar equivalents relative to Compound a4.

Examples of the reaction solvent include methanol, ethanol, acetonitrile, tetrahydrofuran, and dimethylformamide, and one of or a mixture of these can be used.

### General Synthetic Method 2

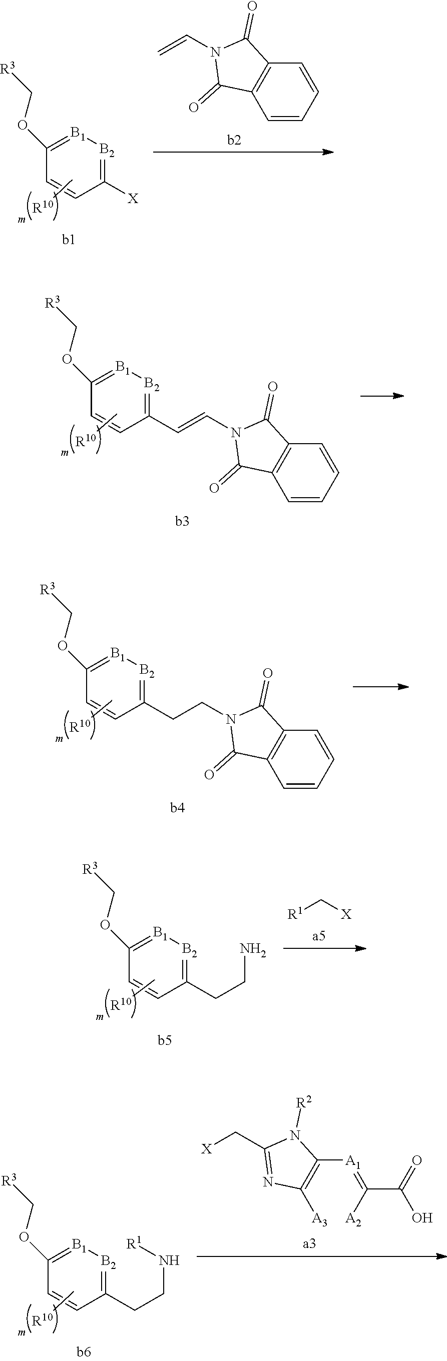

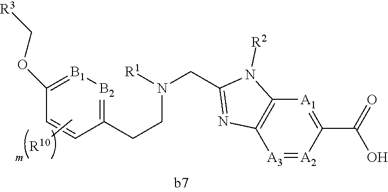

wherein B₁ and B₂ are N or CH, and other symbols have the same meaning as described above.

### Step 1

In the presence of a metal catalyst and a base, tetrabutylammonium bromide or the like is added as necessary, and Compounds b1 and b2 are reacted with each other, whereby Compound b3 can be obtained.

Examples of the metal catalyst include palladium acetate, bis(dibenzylideneacetone)palladium, tetrakis(triphenylphosphine)palladium, bis(triphenylphosphine)palladium (II) dichloride, and bis(tri-tert-butylphosphine)palladium, and the metal catalyst can be used at 0.001 to 0.5 molar equivalents relative to Compound b1.

Examples of the base include dicyclohexylamine, potassium tert-butoxide, sodium carbonate, and potassium carbonate, and the base can be used at 1 to 10 molar equivalents relative to Compound b1.

Compound b2 can be used at 1 to 2 molar equivalents relative to Compound b1.

The reaction temperature may be 20° C. to reflux temperature of solvent, and if necessary, by a microwave irradiation.

The reaction time is 0.1 hours to 48 hours, and preferably 0.5 hours to 12 hours.

Examples of the reaction solvent include tetrahydrofuran, toluene, DMF, dioxane, and water, and one of or a mixture of these can be used.

### Step 2

Compound b4 can be obtained by reacting compound b3 with hydrogen gas in the presence of a metal catalyst.

Examples of the metal catalyst include palladium-carbon, platinum oxide, rhodium-aluminum oxide, and chlorotris(triphenylphosphine)rhodium(I), and the metal catalyst can be used at 0.01 to 100 weight percent relative to Compound b3.

The hydrogen pressure can be 1 to 50 atm. As the hydrogen source, cyclohexene, 1,4-cyclohexadiene, formic acid, ammonium formate, or the like can also be used.

The reaction temperature is 0° C. to the reflux temperature, preferably 20° C. to 40° C.

The reaction time is 0.5 to 72 hours, preferably 1 to 12 hours.

Examples of the reaction solvent include methanol, ethanol, propanol, isopropanol, butanol, tetrahydrofuran, diethyl ether, toluene, ethyl acetate, acetic acid, and water, and one of or a mixture of these can be used.

### Step 3

Compound b5 can be obtained by reacting Compound b4 with hydrazine monohydrate or the like.

Hydrazine monohydrate and the like can be used at 1 to 10 molar equivalent(s) relative to Compound b4.

The reaction temperature is 0° C. to 100° C., preferably 20° C. to 80° C.

The reaction time is 0.5 hours to 24 hours, and preferably 1 hour to 12 hours.

As the reaction solvent, ethanol or the like can be used.

### Step 4

Compound b6 can be obtained by allowing Compound a5 to act on Compound b5 in the presence of a base.

The reaction temperature is 0° C. to the reflux temperature of the solvent.

The reaction time is 0.5 hours to 12 hours, and preferably 1 hour to 6 hours.

Compound a5 can be used at 1 to 20 molar equivalent(s) relative to Compound b5.

Examples of the base include potassium tert-butoxide, sodium tert-butoxide, sodium carbonate, potassium carbonate, and cesium carbonate, and the base can be used at 1 to 20 molar equivalents relative to Compound b5.

Examples of the reaction solvent include methanol, ethanol, acetonitrile, tetrahydrofuran, and dimethylformamide, and one of or a mixture of these can be used.

### Step 5

Compound b7 can be obtained by allowing Compound a3 to act on Compound b6 in the presence of a base.

The reaction temperature is 0° C. to the reflux temperature of the solvent.

The reaction time is 0.5 hours to 12 hours, and preferably 1 hour to 6 hours.

Compound a3 can be used at 0.9 to 3 molar equivalent(s) relative to Compound b6.

Examples of the base include potassium tert-butoxide, sodium tert-butoxide, sodium carbonate, potassium carbonate, and cesium carbonate, and the base can be used at 1 to 5 molar equivalents relative to Compound b6.

Examples of the reaction solvent include methanol, ethanol, acetonitrile, tetrahydrofuran, and dimethylformamide, and one of or a mixture of these can be used.

As the compounds according to the present invention have GLP-1 receptor agonist activity, they are useful as a therapeutic and/or preventive agent for diseases associated with the GLP-1 receptor.

When the term “therapeutic agent and/or prophylactic agent” is used in the present invention, this also encompasses a symptom ameliorating agent.

Examples of the disease associated with the GLP-1 receptor include non-insulin-dependent diabetes mellitus (type 2 diabetes mellitus), hyperglycemia, impaired glucose tolerance, insulin-dependent diabetes mellitus (type 1 diabetes mellitus), diabetic complications, obesity, hypertension, dyslipidemia, arteriosclerosis, myocardial infarction, coronary heart disease, cerebral infarction, non-alcoholic steatohepatitis, Parkinson's disease, and dementia.

In the present invention, “diabetes” means a disease or condition in which the metabolism in the production and utilization of glucose is abnormal due to the inability to maintain an appropriate blood glucose level in the body, and encompasses insulin-dependent diabetes (type 1 diabetes mellitus) and non-insulin-dependent diabetes mellitus (type 2 diabetes mellitus).

“Hyperglycemia” refers to a state in which the plasma glucose level is higher than a normal value (for example, in humans, 80 to 110 mg/dL on an empty stomach) at fasting and after glucose load, and is also one of representative symptoms of diabetes.

Impaired glucose tolerance includes insulin resistant impaired glucose tolerance and insulin hyposecretion.

Diabetic complication means a complication caused by diabetes or hyperglycemia, and may be either an acute complication or a chronic complication. Examples of the “acute complications” include ketoacidosis and infections (for example, skin infections, soft tissue infections, biliary tract infections, respiratory infections, urinary tract infections), and examples of the “chronic complications” include microangiopathies (for example, nephropathy, retinopathy), neurological disorders (for example, a sensory nerve disorder, a motor nerve disorder, or an autonomic nerve disorder), and leg/foot gangrene. Examples of the diabetic complications include diabetic retinopathy, diabetic nephropathy, and diabetic neuropathy. The “coronary heart disease” encompasses myocardial infarction, angina pectoris, and the like.

Examples of the “dementia” include Alzheimer's disease, vascular dementia, and diabetic dementia.

The compound of the present invention has not only GLP-1 receptor agonist activity but also is useful as a medicine, and has any or all of the following excellent characteristics:
a) Inhibitory action against CYP enzymes (e.g., CYP1A2, CYP2C9, CYP2C19, CYP2D6, and CYP3A4) is weak.
b) Satisfactory pharmacokinetics such as high bioavailability and moderate clearance are exhibited.
c) High metabolic stability is exhibited.
d) Irreversible inhibitory action is not exhibited against CYP enzymes (e.g., CYP3A4) within the concentration range of the measurement conditions described in the present specification.
e) Mutagenicity is not exhibited.
f) Low cardiovascular risk is exhibited.
g) Low risk of hematotoxicity is exhibited.
h) High solubility is exhibited.

The pharmaceutical composition of the present invention can be administered by either an oral method or a parenteral method. Examples of a parenteral administration method include percutaneous, subcutaneous, intravenous, intra-arterial, intramuscular, intraperitoneal, transmucosal, inhalation, transnasal, ocular instillation, ear instillation, and intravaginal administration.

In the case of oral administration, the pharmaceutical composition may be prepared into any dosage form that is commonly used, such as a solid preparation for internal use (for example, a tablet, a powder preparation, a granular preparation, a capsule, a pill, or a film preparation), or a liquid preparation for internal use (for example, a suspension, an emulsion, an elixir, a syrup, a limonade, a spirit preparation, an aromatic water preparation, an extraction, a decoction, or a tincture) and administered. The tablet may be a dragee, a film-coated tablet, an enteric-coated tablet, a sustained release tablet, a troche, a sublingual tablet, a buccal tablet, a chewable tablet, or an orally disintegrating tablet; the powder preparation and granular preparation may be dry syrups; and the capsule may be a soft capsule, a microcapsule, or a sustained release capsule.

In the case of parenteral administration, the pharmaceutical composition can be suitably administered in any dosage form that is commonly used, such as an injectable preparation, an infusion, or a preparation for external use (for example, an eye drop, a nasal drop, an ear drop, an aerosol, an inhalant, a lotion, an impregnating agent, a liniment, a gargling agent, an enema, an ointment, a plaster, a jelly, a cream, a patch, a poultice, a powder preparation for external use, or a suppository). The injectable preparation may be an emulsion of O/W type, W/O type, O/W/O type, W/0/W type, or the like.

A pharmaceutical composition can be obtained by mixing an effective amount of the compound of the present invention with various pharmaceutical additives appropriate for the dosage form, such as an excipient, a binder, a disintegrating agent, and a lubricating agent, as necessary. Furthermore, the pharmaceutical composition can be prepared into a pharmaceutical composition for use for a child, an elderly person, a patient with a serious case, or a surgical operation, by appropriately changing the effective amount of the compound of the present invention, the dosage form, and/or various pharmaceutical additives. The pharmaceutical composition for children is preferably administered to patients under the age of 12 or 15. Also, the pharmaceutical composition for children may be administered to patients under 27 days after birth, of 28 days to 23 months after birth, or 2 years to 11 years, of 12 years to 17 years, or of 18 years of age. The pharmaceutical composition for the elderly is preferably administered to patients of 65 years of age or older.

It is desirable to set the amount of administration of the pharmaceutical composition of the present invention, after considering the age and body weight of the patient, the type and degree of the disease, the route of administration, and the like; however, in the case of oral administration, the amount of administration is usually 0.05 to 100 mg/kg/day and is preferably in the range of 0.1 to 10 mg/kg/day. Although varying depending on the administration route, the dose in the case of parenteral administration is within a range of usually 0.005 to 10 mg/kg/day, preferably 0.01 to 1 mg/kg/day. This may be administered once a day or several times a day.

The compound of the present invention can be used in combination of a combined drug, to increase the activity of the compound or reduce the dose of the compound, or the like. At this time, the timing of administration for the compound of the present invention and the concomitant drug is not limited, and these may be administered simultaneously to the target of administration or may be administered with a time difference.

The amount of administration of the concomitant drug can be appropriately selected based on the clinically used dosage. Furthermore, the blending ratio of the compound of the present invention and the concomitant drug can be appropriately selected according to the target of administration, the route of administration, the target disease, symptoms, combination, and the like. For example, when the target of administration is a human, 0.01 to 100 parts by weight of the concomitant drug may be used with respect to 1 part by weight of the compound of the present invention.

The pharmaceutical composition of the present invention can be used in combination with other anti-obesity agent(s) (the pharmaceutical composition comprising compounds having anti-obesity effect, or the medicinal agent for obesity or for the weight management for obesity). For example, a combination treatment with a pharmaceutical composition comprising a compound having an anti-obesity effect and the compound of the present invention can be used for the prevention and/or treatment of obesity or the weight management for obesity. A combination treatment with the pharmaceutical composition comprising the compound of the present invention and a pharmaceutical composition(s) comprising a compound having an anti-obesity effect can be used for the prevention and/or treatment of obesity or the weight management for obesity. Furthermore, a method of treatment by administering the pharmaceutical composition of the invention can be used in combination of the diet therapy, drug therapy, exercise and the like.

### EXAMPLES

Hereinafter, the present invention will be described in more detail by way of Examples, Reference Examples, and Test Examples; however, the present invention is not intended to be limited by these.

Furthermore, abbreviations used in the present specification denote the following meanings.
CHCl₃: chloroform
CDCl₃: deuterated chloroform
MeOH: methanol
DMSO-d₆: deuterated dimethyl sulfoxide
DMSO: dimethyl sulfoxide
DMF: dimethylformamide
THF: tetrahydrofuran
ODS: octadecylsilyl
M: mol/L

### (Method for Identifying Compound)

The NMR analysis obtained in each Example was performed at 400 MHz, and measurement was made using DMSO-d₆, or CDCl₃. Furthermore, when NMR data are shown, there are occasions in which all the measured peaks are not described.

In Examples, “No.” represents compound number, “Structure” means a chemical structure, and “MS” represents a mass in LC/MS (liquid chromatography/mass spectrometry). MS (m/z) can be measured under the following measurement conditions, though the measurement conditions are not limited to these.

### (Measurement Conditions 1)

Column: ACQUITY UPLC BEH C18 (1.7 μm i.d. 2.1×50 mm) (Waters)
Flow rate: 0.8 mL/min
UV detection wavelength: 254 nm
Mobile phase: [A] was 0.1% formic acid-containing aqueous solution, and [B] was 0.1% formic acid-containing acetonitrile solution
Gradient: A linear gradient of 5% to 100% solvent [B] was carried out for 3.5 minutes, and then 100% solvent [B] was maintained for 0.5 minutes.

### (Measurement Conditions 2)

Column: Shim-pack XR-ODS (2.2 μm, i.d. 3.0×50 mm) (Shimadzu)
Flow rate: 1.6 mL/min; UV detection wavelength: 254 nm;
Mobile phase: [A] was 0.1% formic acid-containing aqueous solution, and [B] was 0.1% formic acid-containing acetonitrile solution
Gradient: linear gradient of 10% to 100% solvent [B] was performed in 3 minutes, and 100% solvent [B] was kept for 0.5 minutes.

### (Measurement Conditions 3)

Column: ACQUITY UPLC BEH C18 (1.7 μm i.d. 2.1×50 mm) (Waters)
Flow rate: 0.8 mL/min; UV detection wavelength: 254 nm;
Mobile phase: [A] was 0.1% formic acid-containing aqueous solution, and [B] was 0.1% formic acid-containing acetonitrile solution
Gradient: linear gradient of 5% to 100% solvent [B] for 3.5 minutes was performed, and then 100% solvent [B] was maintained for 0.5 minute.

### (Measurement Conditions 4)

Column: ACQUITY UPLC BEH C18 (1.7 μm i.d. 2.1×50 mm) (Waters)
Flow rate: 0.8 mL/min
UV detection wavelength: 254 nm
Mobile phase: [A] is 10 mM ammonium carbonate in aqueous solution, and [B] is acetonitrile
Gradient: A linear gradient of 5% to 100% solvent [B] was carried out for 3.5 minutes, and then 100% solvent [B] was maintained for 0.5 minutes.

### (Measurement Conditions 5)

Column: L-column 2 ODS (3 μm i.d. 3×50 mm) (manufactured by Chemicals Evaluation and Research Institute)
Flow rate: 1.5 mL/min
UV detection wavelength: 220 nm
Mobile phase: [A] was 0.05% trifluoroacetic acid-containing aqueous solution, and [B] was 0.05% trifluoroacetic acid-containing acetonitrile solution
Gradient: linear gradient of 5% to 95% solvent [B] was performed in 3.5 minutes, and 95% solvent [B] was kept for 2 minutes.

### Example 1

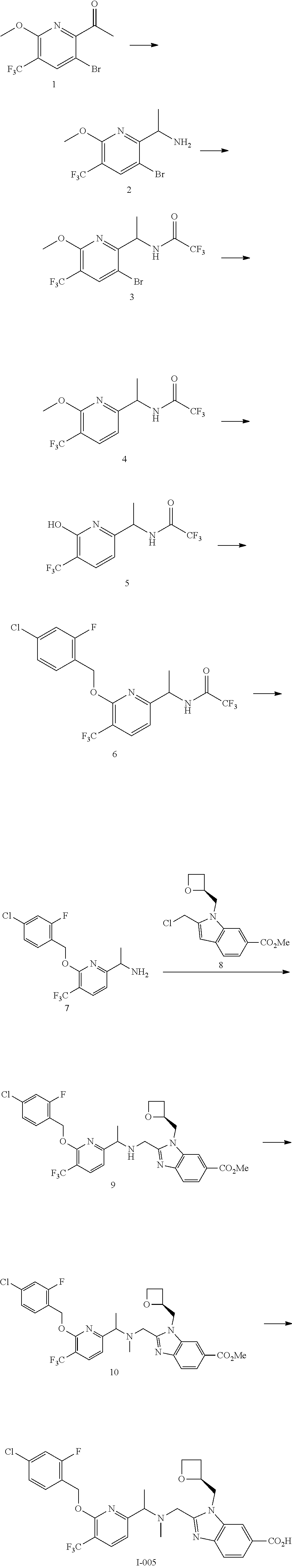

### Step 1

Ammonium acetate (3.88 g, 50.3 mmol) and sodium cyanoborohydride (253 mg, 4.03 mmol) were added to a methanol (30 mL) solution of Compound 1 (1.50 g, 5.03 mmol), and the mixture was stirred at room temperature for 19 hours. The reaction solution was concentrated, then water and a 1 M aqueous sodium hydroxide solution were added, and the mixture was extracted with dichloromethane. The solvent was evaporated under reduced pressure, and the obtained residue was purified by silica gel column chromatography (chloroform-methanol) to give Compound 2 (522.8 mg, yield 35%).

MS (m/z)=299.1 [M+H]+

¹H-NMR (DMSO-D₆) δ: 8.23 (1H, s), 4.29 (1H, q, J=6.7 Hz), 4.03 (3H, s), 1.25 (3H, d, J=6.7 Hz).

### Step 2

Trifluoroacetic anhydride (404 μL, 2.86 mmol) was added to a dichloromethane (6 mL) solution of Compound 2 (571.2 mg, 1.91 mmol) under ice cooling, then the mixture was stirred at room temperature for 3 hours. The solvent was evaporated under reduced pressure to give Compound 3 (739.2 mg, yield 98%).

MS (m/z)=395.1 [M+H]+

¹H-NMR (CDCl₃) δ: 8.02 (1H, s), 7.68 (1H, s), 5.51-5.44 (1H, m), 4.06 (3H, s), 1.53 (3H, d, J=6.7 Hz).

### Step 3

10w % palladium carbon (199 mg, 0.187 mmol) was added to a methanol (10 mL) solution of Compound 3 (739.2 mg, 1.87 mmol), then degassed under reduced pressure, and substituted with hydrogen. The reaction solution was stirred at room temperature for 57 hours under a nitrogen atmosphere, and then filtered through Celite (registered trademark), and the solvent was evaporated under reduced pressure to give compound 4 (620.3 mg, yield 104%).

MS (m/z)=317.2 [M+H]+

¹H-NMR (CDCl₃) δ: 7.88 (2H, d, J=7.7 Hz), 7.61 (1H, s), 6.93 (1H, d, J=7.7 Hz), 5.18-5.11 (1H, m), 4.07 (3H, s), 1.56 (3H, d, J=6.8 Hz).

### Step 4

Sodium iodide (588 mg, 3.92 mmol) and chlorotrimethylsilane (426 mg, 3.92 mmol) were added to an acetonitrile (10 mL) solution of Compound 4 (620.3 mg, 1.96 mmol), then the mixture was stirred at room temperature for 1 hour. Sodium bicarbonate water and aqueous sodium thiosulfate solution were added to the reaction solution, and then the mixture was extracted with ethyl acetate. The obtained organic layer was dried over anhydrous sodium sulfate, and then the solvent was evaporated under reduced pressure to give compound 5 (499.4 mg, 84% yield).

MS (m/z)=303.2 [M+H]+

¹H-NMR (CDCl₃) δ: 8.04 (1H, d, J=8.4 Hz), 7.90 (1H, d, J=7.4 Hz), 6.50 (1H, d, J=7.4 Hz), 5.17-5.09 (1H, m), 1.66 (3H, d, J=7.3 Hz).

### Step 5

To a 1,4 dioxane (20 mL) solution of Compound 5 (499.4 mg, 1.65 mmol), silver carbonate (911 mg, 3.31 mmol) and 1-bromomethyl-4-chloro-2 fluorobenzene (406 mg, 1.82 mmol) were added, and the mixture was stirred at 65° C. for 3.5 hours. Insolubles in the reaction solution were removed by filtration, and the solvent was evaporated under reduced pressure. The obtained residue was purified by silica gel column chromatography (hexane-ethyl acetate) to give Compound 6 (778.8 mg, yield 100%).

MS (m/z)=445.3 [M+H]+

¹H-NMR (CDCl₃) δ: 7.90 (1H, d, J=7.7 Hz), 7.44-7.42 (1H, m), 7.37 (1H, d, J=6.1 Hz), 7.17-7.09 (2H, m), 6.94 (1H, d, J=7.7 Hz), 5.55 (2H, s), 5.18-5.11 (1H, m), 1.49 (3H, d, J=6.8 Hz).

### Step 6

A 1 M aqueous sodium hydroxide solution (3.5 mL, 3.50 mmol) was added to a methanol (10 mL)-tetrahydrofuran (10 mL) mixture solution of Compound 6 (778.8 mg, 1.75 mmol), then the mixture was stirred at room temperature for 20 hours. Water was added to the reaction solution, and the solution was extracted with ethyl acetate. The obtained organic layer was dried over anhydrous sodium sulfate, and then the solvent was evaporated under reduced pressure to give compound 7 (538.1 mg, 88% yield).

MS (m/z)=349.2 [M+H]+

¹H-NMR (CDCl₃) δ: 7.83 (1H, d, J=7.7 Hz), 7.47-7.45 (1H, m), 7.15-7.11 (2H, m), 6.96 (1H, d, J=7.8 Hz), 5.55 (2H, s), 4.09-4.02 (1H, m), 1.39 (3H, d, J=6.7 Hz).

### Step 7

Potassium carbonate (85.0 mg, 0.618 mmol) was added to an acetonitrile (3 mL) solution of Compound 7 (107.8 mg, 0.309 mmol) and Compound 8 (96.0 mg, 0.325 mmol), and the mixture was stirred at 60° C. for 8 hours. The reaction solution was cooled to room temperature, water was added thereto, and the solution was extracted with ethyl acetate. The obtained organic layer was dried over anhydrous sodium sulfate, and the solvent was evaporated under reduced pressure to give a mixture (212.5 mg) of Compound 9 and impurities.

MS (m/z)=607.4 [M+H]+

### Step 8

Methyl iodide (50 μL, 0.80 mmol) and potassium carbonate (42.8 mg, 0.309 mmol) were added to an acetonitrile (5 mL) solution of a mixture (93.9 mg) of Compound 9 and impurities, and the mixture was stirred at room temperature for 5 hours. Water was added to the reaction solution, and the solution was extracted with ethyl acetate. After the organic layer was washed with water and dried over anhydrous sodium sulfate, the solvent was evaporated under reduced pressure, and the obtained residue was purified by silica gel column chromatography (hexane-ethyl acetate) to give a mixture (27.1 mg, yield 28%) of Compound 10 and impurities.

MS (m/z)=621.4 [M+H]+

### Step 9

A 1 M aqueous sodium hydroxide solution (0.202 mL, 0.202 mmol) was added to a methanol (2 mL)-tetrahydrofuran (2 mL) mixture solution of Compound 10 (25.1 mg, 0.040 mmol), then the mixture was stirred at 45° C. for 10.5 hours. The reaction solution was cooled to room temperature, and then, 2M hydrochloric acid was added to adjust the pH to 4, and the mixture was extracted with ethyl acetate. The obtained organic layer was dried over anhydrous sodium sulfate, then the solvent was evaporated under reduced pressure, and the obtained residue was purified by reverse phase column chromatography (acetonitrile-water) to give Compound I-005 (diastereomeric mixture, 15.1 mg, 62% yield).

MS (m/z)=607.4 [M+H]+

¹H-NMR (DMSO-D₆) δ: 8.21 (1H, s), 8.12 (1H, dd, J=7.6, 4.5 Hz), 7.78 (1H, d, J=7.9 Hz), 7.60 (1H, d, J=8.2 Hz), 7.54-7.52 (1H, m), 7.47-7.45 (1H, m), 7.30-7.28 (1H, m), 7.24-7.23 (1H, m), 5.57 (2H, s), 4.98-4.96 (1H, m), 4.71-4.69 (1H, m), 4.56-4.53 (1H, m), 4.44-4.42 (1H, m), 4.27-4.25 (1H, m), 3.98-3.81 (3H, m), 2.62-2.60 (1H, m), 2.30 (1H, s), 2.09 (3H, s), 1.42-1.40 (3H, m).

### Example 2

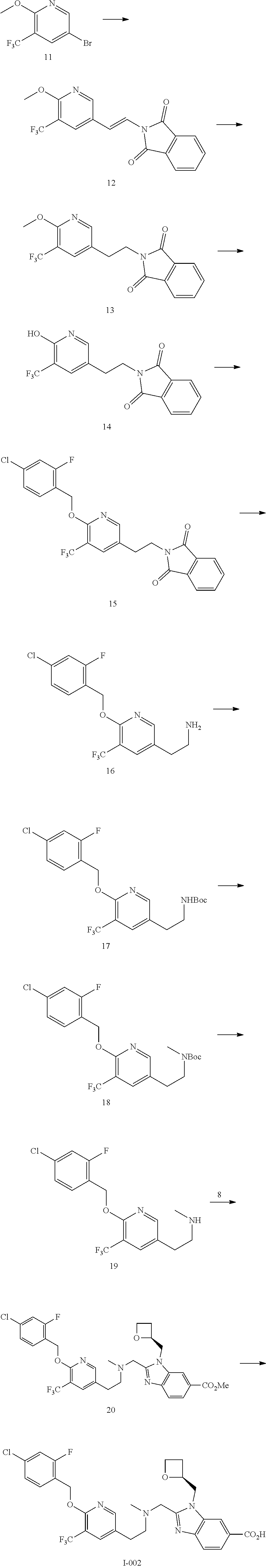

### Step 1

N,N-dimethylcyclohexylmethylamine was added to a DMF (16 mL) solution of Compound 11 (2 g, 7.81 mmol), vinyl phthalimide (1.38 g, 7.81 mmol), palladium acetate (175 mg, 0.781 mmol), and tetrabutylammonium bromide (2.52 g, 7.81 mmol), and the mixture was heated at 104° C. for 90 minutes under a nitrogen atmosphere. The reaction solution was cooled to room temperature, and then water was added and stirred. The obtained solid was collected by filtration and washed with water and isopropyl ether to give Compound 12 (1.95 g, yield 72%).

MS (m/z)=349.05 [M+H]+

¹H-NMR (CDCl₃) δ: 8.35 (1H, s), 7.99 (1H, s), 7.93 (2H, dd, J=5.4, 3.0 Hz), 7.79 (2H, dd, J=5.4, 3.0 Hz), 7.63 (1H, d, J=15.2 Hz), 7.30 (1H, d, J=15.2 Hz), 4.06 (3H, s).

### Step 2

10% palladium carbon (water content: 50%) was added to a methanol (39 mL)-THF (39 mL) mixture solution of Compound 12 (1.95 g, 5.60 mmol), and the mixture was stirred for 19 hours under a hydrogen atmosphere. The reaction solution was filtered through Celite (registered trademark), then the solvent was evaporated under reduced pressure, and the obtained crude product was washed with isopropyl ether (10 mL) to give Compound 13 (1.75 g, yield 89%).

MS (m/z)=351.0 [M+H]+

¹H-NMR (CDCl₃) δ: 8.16 (1H, s), 7.84 (2H, dd, J=5.5, 3.1 Hz), 7.77-7.74 (1H, m), 7.73 (2H, dd, J=5.5, 3.1 Hz), 4.00 (3H, s), 3.91 (2H, t, J=7.5 Hz), 2.98 (2H, t, J=7.5 Hz).

### Steps 3 and 4

Sodium iodide (450 mg, 3.00 mmol) and trimethylsilyl chloride (383 μL, 3.00 mmol) were added to an acetonitrile (3.5 mL) solution of Compound 13 (350 mg, 1.00 mmol), and the mixture was stirred at 45° C. for 90 minutes. Saturated sodium bicarbonate water and aqueous sodium thiosulfate solution were added to the reaction solution, and the solution was extracted with ethyl acetate, then the solvent was evaporated under reduced pressure to give Compound 14.

Silver carbonate (414 mg, 1.50 mmol) and 1-bromo-4-chloro-2-fluorobenzene (268 mg, 1.20 mmol) were added to a dioxane (3.5 mL) solution of Compound 4 thus obtained, and the mixture was stirred at 65° C. for 3 hours and 30 minutes. The reaction solution was filtered, washed with ethyl acetate, and then the obtained solution was concentrated under a reduced pressure. The obtained solid was washed with isopropyl ether (6 mL) to give Compound 15 (384 mg, yield 80%).

MS (m/z)=479.0 [M+H]+

¹H-NMR (CDCl₃) δ: 8.16 (1H, s), 7.84 (2H, dd, J=5.5, 3.1 Hz), 7.81-7.78 (1H, m), 7.73 (2H, dd, J=5.4, 3.0 Hz), 7.44 (1H, t, J=8.0 Hz), 7.16-7.07 (2H, m), 5.49 (2H, s), 3.91 (2H, t, J=7.5 Hz), 2.99 (2H, t, J=7.5 Hz).

### Steps 5 and 6

Hydrazine monohydrate (194 μL, 4.00 mmol) was added to an ethanol (7.7 mL) solution of Compound 15 (384 mg, 0.800 mmol), and the mixture was stirred at 50° C. for 90 minutes. Water was added to the reaction solution. The mixture was extracted with chloroform. The solvent of the organic layer was evaporated under reduced pressure to give Compound 16.

Di-tert-butyl dicarbonate (223 μL, 0.960 mmol) was added to Compound 16 thus obtained, and the mixture was stirred for 1 hour. The reaction solution was concentrated under reduced pressure, and the obtained residue was purified by silica gel column chromatography (hexane-ethyl acetate) to give Compound 17 (341 mg, yield 95%).

MS (m/z)=449.0 [M+H]+

¹H-NMR (CDCl₃) δ: 8.14 (1H, s), 7.72 (1H, s), 7.46 (1H, t, J=8.0 Hz), 7.17-7.08 (2H, m), 5.51 (2H, s), 4.57 (11, s), 3.35 (2H, d, J=6.7 Hz), 2.79 (2H, t, J=6.8 Hz), 1.43 (9H, s).

### Steps 7 to 9

60 wt % sodium hydride (6.68 mmol) and methyl iodide (13.9 μL, 0.223 mmol) were added to a DMF (200 μL) solvent of Compound 17 (50 mg, 0.111 mmol), and the mixture was stirred at room temperature for 1 hour. A saturated aqueous sodium hydrogen chloride solution was added to the reaction solution, and the mixture was extracted with ethyl acetate. The solvent of the organic layer was evaporated under reduced pressure to give Compound 18.

Dichloromethane (0.500 mL) and TFA (129 μL, 1.67 mmol) were added to Compound 18 thus obtained, and the mixture was stirred at room temperature for 90 minutes. The reaction solution was concentrated under reduced pressure, and then an azeotrope with toluene was formed to give Compound 19.

Compound 8 (32.8 mg, 0.111 mmol) and potassium carbonate (31 mg, 0.223 mmol) were added to an acetonitrile (1 mL) solution of Compound 19 thus obtained, and the mixture was stirred at room temperature for 2 hours. A saturated aqueous ammonium chloride solution was added to the reaction solution, and the mixture was extracted with ethyl acetate. The solvent of the organic layer was evaporated under reduced pressure, and the obtained residue was purified by silica gel column chromatography (hexane-ethyl acetate) to give Compound 20 (48.6 mg, yield 70%).

MS (m/z)=620.95 [M+H]+

¹H-NMR (CDCl₃) δ: 8.03 (1H, s), 8.02 (1H, s), 7.96 (1H, d, J=8.5 Hz), 7.73 (1H, d, J=8.5 Hz), 7.63 (1H, s), 7.46 (1H, t, J=8.0 Hz), 7.19-7.09 (2H, m), 5.47 (2H, s), 5.06-4.98 (1H, m), 4.58-4.51 (1H, m), 4.43-4.34 (1H, m), 4.34-4.27 (1H, m), 4.27-4.18 (1, m), 4.03-3.95 (2H, m), 3.92 (3H, s), 2.81-2.70 (1H, m), 2.81-2.70 (1H, m), 2.68-2.58 (1H, m), 2.35 (3H, s), 2.29-2.18 (1H, m).

### Step 10

A 2 M aqueous sodium hydroxide solution (114 μL, 0.227 mmol) was added to a THF-MeOH (1:1, 470 μL) mixture solution of Compound 20 (47 mg, 0.076 mmol), and the mixture was stirred at room temperature for 3 hours. The reaction solution was concentrated under reduced pressure, and the obtained residue was purified by silica gel column chromatography (chloroform-methanol) to give Compound I-002 (36.7 mg, yield 80%).

MS (m/z)=607.1 [M+H]+

¹H-NMR (CDCl₃) δ: 8.12 (1H, s), 8.07 (1H, d, J=8.5 Hz), 8.05 (1H, s), 7.83 (1H, d, J=8.5 Hz), 7.65 (1H, s), 7.46 (1H, t, J=8.0 Hz), 7.17-7.07 (2H, m), 5.47 (2H, s), 5.09-5.01 (1H, m), 4.61-4.50 (1H, m), 4.49-4.39 (1H, m), 4.38-4.31 (1H, m), 4.30-4.22 (1H, m), 4.10-4.01 (2H, m), 2.81-2.73 (1H, m), 2.81-2.73 (1H, m), 2.71-2.59 (1H, m), 2.35 (3H, s), 2.32-2.21 (1H, m).

The following compounds were also synthesized in the same manner by using the general synthesis method described above or the synthesis method described in Examples. I-003 and 1-004 in the table are diastereomeric mixtures.

TABLE 1

| No. | Structure |
|---|---|
| I-001 | 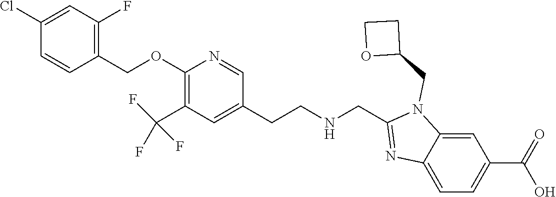 [SMILES: O=C(O)c1ccc2nc(CNCCc3cnc(OCc4ccc(Cl)cc4F)c(C(F)(F)F)c3)n(C[C@@H]3CCO3)c2c1] |
| I-003 | 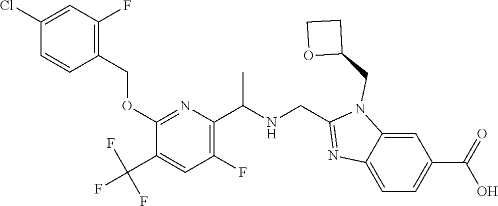 [SMILES: CC(NCc1nc2ccc(C(=O)O)cc2n1C[C@@H]1CCO1)c1nc(OCc2ccc(Cl)cc2F)c(C(F)(F)F)cc1F] |
| I-004 | 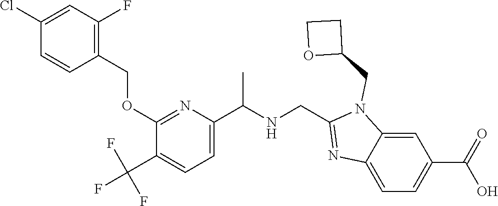 [SMILES: CC(NCc1nc2ccc(C(=O)O)cc2n1C[C@@H]1CCO1)c1ccc(C(F)(F)F)c(OCc2ccc(Cl)cc2F)n1] |
| I-006 | 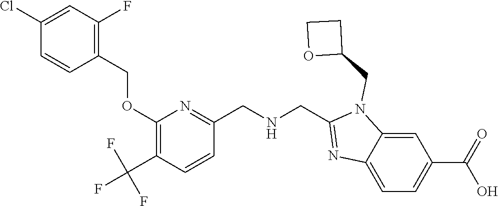 [SMILES: O=C(O)c1ccc2nc(CNCc3ccc(C(F)(F)F)c(OCc4ccc(Cl)cc4F)n3)n(C[C@@H]3CCO3)c2c1] |
| I-007 | 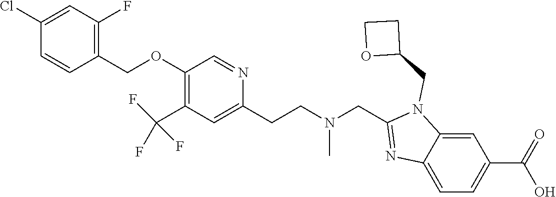 [SMILES: CN(CCc1cc(C(F)(F)F)c(OCc2ccc(Cl)cc2F)cn1)Cc1nc2ccc(C(=O)O)cc2n1C[C@@H]1CCO1] |

TABLE 2

| No. | MS | Charge |
|---|---|---|
| I-001 | 593.05 | M + H |
| I-003 | 611.43 | M + H |
| I-004 | 593.43 | M + H |
| I-006 | 579.42 | M + H |
| I-007 | 607.25 | M + H |

Biological Test Examples for the compounds of the present invention will be described below. The compounds of the present invention can be tested essentially as described in test examples below.

The compound represented by formula (I) according to the present invention has a GLP-1 receptor agonist activity effect.

Specifically, in the evaluation method described below, the EC₅₀ value is preferably 5000 nM or less, more preferably 1000 nM or less, even more preferably 100 nM or less.

### Test Example 1: Measurement of GLP-1 Receptor Agonist Activity

### Cell Culture

Human GLP-1 receptor stably expressing cells (hGLP-1R/CHO-K1 cells) are cultured in an α-MEM medium (Sigma) containing 10% FBS (Hyclone), 2% GlutaMAX (Gibco), 1% G418 (Nacalai Tesque), and 1% Penicillin-Streptomycin Mixed Solution (Sigma) at 37° C. under 5% CO₂ conditions, and recovered by treating a 10 fold diluted 5.0 g/1-trypsin/5.3 mmol/1-EDTA solution (Nacalai Tesque) and cryopreserved.

cAMP Assay

A DMSO solution containing the compound of the present invention or human GLP-1 (7-36) (Phoenix Pharmaceuticals) is dispensed into a 384-well microplate (Greiner) at 62.5 nL/well, and in addition, 400 μM Forskolin (Nacalai Tesque) is dispensed therein at 7.5 nL/well. Subsequently, the frozen GLP-1 R/CHO-K1 cells are thawed in a thermostatic bath at 37° C., suspended in HBSS buffer (GIBCO) containing 0.1% BSA (Sigma), 20 mM HEPES, 0.1 mM IBMX (Sigma), and 0.2 mM RO20-1724 (Calbiochem) at 2×10⁴ cells/mL, and the cell suspension is added at 6 μL/well. The solution was incubated at 37° C. for 1 hour and intracellular cAMP concentration is measured using cAMP Gs dynamic kit (Cisbio) according to the protocol attached to the product. Specifically, a cAMP-d2/Anti-cAMP-Cryptate (1/1) mixture solution is added in a total amount of 6 μL/well, and is incubated at room temperature for 1 hour, and then, time-resolved fluorescence is measured using PHERAstar (BMG Labtech).

Assuming that the cAMP concentration when human GLP-1 (7-36) is dispensed to a final concentration of 2 nM is 100% and the cAMP concentration when only DMSO is dispensed is 0%, the 50% effective concentration (EC₅₀) and the maximum effect (Emax) of the compound of the present invention are calculated using an increase in the cAMP concentration as an index, using TIBCO Spotfire (TIBCO Software). The dilution concentration or the dilution solvent are changed as necessary.

The compounds of the present invention were tested essentially as described above. EC₅₀ and Emax of each compound of the present invention are shown in the following table.

TABLE 3

| No. | EC50_nM | Emax_% |
|---|---|---|
| I-002 | 2300 | 52 |
| I-003 | 354 | 65.3 |
| I-004 | 1060 | 60 |
| I-005 | 86 | 74.7 |
| I-006 | 2830 | 55 |

From the above results, since the compound of the present invention exhibited GLP-1 receptor agonist activity, the compound is expected to have an effect as a therapeutic or prophylactic agent for a disease involving the GLP-1 receptor.

### Test Example 2: Metabolism Stability Test

Using commercially available pooled human liver microsomes, a compound of the present invention is reacted for a constant time, and a remaining rate is calculated by comparing a reacted sample and an unreacted sample, thereby, a degree of metabolism in liver is assessed.

A reaction is performed (oxidative reaction) at 37° C. for 0 minute or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human liver microsomes. After the reaction, 70 μL of the reaction solution is added to 140 μL of a methanol/acetonitrile=1/1 (v/v), mixed and centrifuged at 3000 rpm for 15 minutes. The compound of the present invention in the centrifuged supernatant is quantified by LC/MS/MS or solid-phase extraction (SPE)/MS. The ratio of the amount of the compound after the reaction, with respect to the amount of the compound at 0 minutes of the reaction defined as 100%, is shown as the residual rate. The dilution concentration or the dilution solvent are changed as necessary.

The compounds of the present invention can be tested essentially as described above.

### Test Example 3: Solubility Test

The solubility of the compound of the present invention is determined under 1% DMSO addition conditions. A 10 mmol/L solution of the compound is prepared with DMSO, and 2 μL of the solution of the compound of the present invention is added, respectively, to 198 μL of the second fluid for the dissolution test in the Japanese Pharmacopeia (JP 17). The mixture is shaken for 3 hours at a room temperature, and the mixture is vacuum-filtered. The filtrate is 100-fold diluted with methanol/acetonitrile/water=1/1/2 (V/V/V), and the compound concentration in the filtrate is measured with LC/MS/MS by the absolute calibration method.

The compounds of the present invention can be tested essentially as described above.

### Test Example 4: CYP Inhibition Test

Using commercially available pooled human liver microsome, and employing, as markers, 7-ethoxyresorufin O-deethylation (CYP1A2), tolbutamide methyl-hydroxylation (CYP2C9), mephenytoin 4′-hydroxylation (CYP2C19), dextromethorphan O-demethylation (CYP2D6), and terfenadine hydroxylation (CYP3A4) as typical substrate metabolism reactions of human main five CYP enzyme forms (CYP1A2, 2C9, 2C19, 2D6, 3A4), an inhibitory degree of each metabolite production amount by the compound of the present invention was assessed.

The reaction conditions are as follows: substrate, 0.5 μmol/L ethoxyresorufin (CYP1A2), 100 μmol/L tolbutamide (CYP2C9), 30 μmol/L or 50 μmol/L S-mephenytoin (CYP2C19), 5 μmol/L dextromethorphan (CYP2D6), 1 μmol/L terfenadine (CYP3A4); reaction time, 15 minutes; reaction temperature, 37° C.; enzyme, 0.2 mg protein/mL of pooled human liver microsome; concentrations of the compound of the present invention, 1, 5, 10, 20 μmol/L (four points).

Each five kinds of substrates, human liver microsomes, or compound of the present invention in 50 mmol/L Hepes buffer are added to a 96-well plate at the composition as described above, and NADPH, as a cofactor is added to initiate metabolism reactions. After reaction at 37° C. for 15 minutes, the reaction is terminated by the addition of a solution of methanol/acetonitrile=1/1 (V/V). After the centrifugation at 3000 rpm for 15 minutes, resorufin (CYP1A2 metabolite) in the supernatant is quantified by a fluorescent multilabel counter or LC/MS/MS and hydroxytolbutamide (CYP2C9 metabolite), 4′ hydroxymephenytoin (CYP2C19 metabolite), dextromethorphan (CYP2D6 metabolite), and terfenadine alcohol metabolite (CYP3A4 metabolite) are quantified by LC/MS/MS.

The sample in which only DMSO as a solvent dissolving a drug is added to a reaction system is adopted as a control (100%). Remaining activity (%) is calculated and IC₅₀ is calculated by reverse presumption by a logistic model using a concentration and an inhibition rate.

The compounds of the present invention can be tested essentially as described above.

### Test Example 5: BA Test

Materials and methods for experiments to evaluate oral absorption
(1) Experimental animals: Mice or SD rats are used.
(2) Rearing condition: Mice or SD rats are allowed free access to solid feed and sterilized tap water.
(3) Setting of dosage and grouping: Oral administration and Intravenous administration were performed with the predetermined dosages. Grouping is set as below. (Dosage can be changed per compound)
Oral administration 2 to 60 μmol/kg or 1 to 30 mg/kg (n=2 to 3)
Intravenous administration 1 to 20 μmol/kg or 0.5 to 10 mg/kg (n=2 to 3)
(4) Preparation of administration solutions: Oral administration is performed as solution or suspension. Intravenous administration is performed after solubilization.
(5) Routes of administration: Oral administration is performed mandatory into the stomach by oral sonde. Intravenous administration is performed from caudal vein or femoral vein by a needled syringe.
(6) Evaluation items: Blood was collected over time, and the plasma concentration of the compound of the present invention is measured using LC/MS/MS.
(7) Statistical analysis: An area under plasma concentration-time curve (AUC) is calculated as to change in the concentration of the compound of the present invention in plasma by the moment analysis method, and bioavailability (BA) of the compound of the present invention is calculated from a dose ratio and an AUC ratio between the oral administration group and the intravenous administration group.

The compounds of the present invention can be tested essentially as described above.

### Test Example 6: Clearance Evaluation Test

### Materials and Methods for Experiments

(1) Experimental animals: SD rats are used.
(2) Rearing condition: SD rats are allowed free access to solid feed and sterilized tap water.
(3) Setting of dosage and grouping: Intravenous administration was performed with the predetermined dosage. Grouping is set as below.
Intravenous administration 1 μmol/kg (n=2)
(4) Preparation of administration solutions: Administration is performed after solubilization by using dimethyl sulfoxide/propylene glycol=1/1 as the solvent.
(5) Routes of administration: Intravenous administration is performed from caudal vein by a needled syringe.
(6) Evaluation items: Blood is collected serially and concentration of a compound according to the present invention in plasma is measured by LC/MS/MS.
(7) Statistical analysis: About transition of concentration of a compound according to the present invention in plasma, total clearance (CLtot) of a compound according to the present invention is calculated by the moment analysis method. The dilution concentration or the dilution solvent are changed as necessary. The compounds of the present invention can be tested essentially as described above.

### Test Example 7: CYP3A4 (MDZ) MBI Test

CYP3A4 (MDZ) MBI test is a test of investigating mechanism based inhibition (MBI) potential on CYP3A4 by the enhancement of inhibitory degree of a metabolic reaction caused by the compound of the present invention. The inhibition of CYP3A4 is evaluated in pooled human liver microsomes by using the 1-hydroxylation reaction of midazolam (MDZ) as an index.

The reaction conditions are as follows: substrate, 10 μmol/L MDZ; pre-reaction time, 0 or 30 minutes; substrate reaction time, 2 minutes; reaction temperature, 37° C.; protein content of pooled human liver microsomes, at pre-reaction time 0.5 mg/mL, at reaction time 0.05 mg/mL (at 10-fold dilution); concentrations of the compound of the present invention, 0.83, 5, 10, 20 μmol/L (four points).

Pooled human liver microsomes and a solution of the compound of the present invention in K-Pi buffer (pH 7.4) as a pre-reaction solution are added to a 96-well plate at the composition of the pre-reaction. A part of pre-reaction solution is transferred to another 96-well plate, and 1/10 diluted by K-Pi buffer containing a substrate. NADPH as a co-factor is added to initiate a reaction as a marker reaction (without preincubation). After a predetermined time of a reaction, methanol/acetonitrile=1/1 (V/V) solution is added to stop the reaction. In addition, NADPH is added to a remaining pre-reaction solution to initiate a pre-reaction (with preincubation). After a predetermined time of a pre-reaction, a part is transferred to another plate, and 1/10 diluted by K-Pi buffer containing a substrate to initiate a reaction as a marker reaction. After reaction for a given time, the reaction is terminated by the addition of a solution of methanol/acetonitrile=1/1 (V/V). Each plate where the index reaction has been performed is centrifuged at 3000 rpm for 15 minutes. Then, midazolam 1-hydroxide in the centrifugation supernatants is quantified by LC/MS/MS.

The sample in which only DMSO as a solvent dissolving the compound of the present invention is added to a reaction system is adopted as a control (100%). Remaining activity (%) is calculated at each concentration of the compound of the present invention compared to control, and IC value is calculated by reverse-presumption by a logistic model using a concentration and an inhibition rate. Shifted IC value is calculated as “IC of preincubation at 0 min/IC of preincubation at 30 min”. When a shifted IC is 1.5 or more, this is defined as positive. When a shifted IC is 1.0 or less, this is defined as negative.

The compounds of the present invention can be tested essentially as described above.

### Test Example 8: Powder Solubility Test

Appropriate quantity of the compound of the present invention is put in suitable containers. 200 μL of JP-1 fluid (water is added to 2.0 g of sodium chloride and 7.0 mL of hydrochloric acid to reach 1000 mL), 200 μL of JP-2 fluid (1.70 g of sodium dihydrogen phosphate and 1.775 g of anhydrous disodium hydrogen phosphate are dissolved in 1000 mL of water to obtain a buffer solution of pH 6.8 to 6.9), or 20 mmol/L sodium taurocholate (TCA)/JP-2 fluid (JP-2 fluid is added to 1.08 g of TCA to reach 100 mL) is independently added to each container. When the total amount is dissolved after adding the test reagent, the compound of the present invention is added appropriately. The containers are hermetically sealed, shaken at 37° C. for 1 hour, and then filtered. Each filtrate is diluted 2-fold by addition of 100 μL of methanol to 100 μL of the filtrate. The dilution rate is changed as necessary. After checking that there is no bubble and precipitate, the container is sealed and shaken. The compound of the present invention is measured using HPLC by absolute calibration curve method.

The compounds of the present invention can be tested essentially as described above.

### Test Example 9: Fluctuation Ames Test

The compound of the present invention is evaluated for its mutagenicity. Cryopreserved Salmonella typhimurium (TA98 strain and TA100 strain), 20 μL, was inoculated to 10 mL of a liquid nutrient medium (2.5% Oxoid nutrient broth No. 2) and shake-precultured at 37° C. for 10 hours. The bacterial solution of the TA98 strain, 8.0 to 11.0 mL, is centrifuged (2000×g, 10 min) to remove the culture solution. The bacteria was suspended in 8.0 to 11.0 mL of a Micro F buffer (K₂HPO₄: 3.5 g/L, KH₂PO₄: 1 g/L, (NH₄)₂SO₄: 1 g/L, trisodium citrate dihydrate: 0.25 g/L, MgSO₄·7H₂O: 0.1 g/L), the suspension was added to 120 mL of an Exposure medium (Micro F buffer containing Biotin: 8 μg/mL, histidine: 0.2 μg/mL, glucose: 8 mg/mL). The TA100 strain was added to 120 mL of the Exposure medium relative to 3.1 mL of the bacterial solution to prepare a test bacterial solution. 12 μL each of a DMSO solution of the compound of the present invention (several serial dilutions from maximum dose 50 mg/mL at 2- to 3-fold common ratio), DMSO as a negative control, and 50 μg/mL of a 4-nitroquinoline-1-oxide DMSO solution for the TA98 strain and 0.25 μg/mL of a 2-(2-furyl)-3-(5-nitro-2-furyl)acrylamide DMSO solution for the TA100 strain under non-metabolic activation conditions or 40 μg/mL of a 2-aminoanthracene DMSO solution for the TA98 strain and 20 μg/mL of a 2-aminoanthracene DMSO solution for the TA100 strain under metabolic activation conditions as a positive control, and 588 μL of the test bacterial solution (a mixed solution of 498 μL of the test bacterial solution and 90 μL of S9 mix under metabolic activation conditions) are mixed, and the mixture is shake-cultured at 37° C. for 90 minutes. A microbial cell solution exposed to the compound of the present invention and an Indicator medium (Micro F buffer containing 8 μg/mL biotin, 0.2 μg/mL histidine, 8 mg/mL glucose, and 37.5 μg/mL Bromo Cresol Purple) are mixed at a ratio of 23: 115, and 50 μL of the microbial cell solution containing Indicator, 2760 μL in total, is dispensed to microplate 48 wells/dose, and this is incubated at 37° C. for 3 days. The color of the solution in a well containing a bacterium that has acquired growth ability due to a mutation in amino acid (histidine) synthase gene is changed from purple to yellow depending on pH change. Therefore, yellow-colored wells with bacterial growth among the 48 wells per dose are counted and evaluated by comparison with the negative control group. Negativity to mutagenicity is indicated by (−), and positivity thereto is indicated by (+).

The compounds of the present invention can be tested essentially as described above.

### Test Example 10: hERG Test

For the purpose of evaluating the compound of the present invention for the risk of electrocardiogram QT interval prolongation, the effect of the compound of the present invention on delayed rectifier K⁺ current (I_{Kr}) which plays an important role in a ventricular repolarization process is studied using CHO cells caused to express a human ether-a-go-go related gene (hERG) channel.

The cells are kept at a membrane potential of −80 mV by the whole cell patch clamp method using a fully automated patch clamp system (QPatch; Sophion Bioscience A/S), given a leak potential of −50 mV, and then given depolarization stimulation of +20 mV for 2 seconds and further repolarization stimulation of −50 mV for 2 seconds. I_{Kr} induced by this procedure is recorded. An extracellular fluid (NaCl: 145 mmol/L, KCl: 4 mmol/L, CaCl₂): 2 mmol/L, MgCl₂: 1 mmol/L, glucose: 10 mmol/L, HEPES (4-(2-hydroxyethyl)-1-piperazineethanesulfonic acid): 10 mmol/L, pH=7.4) adjusted to 0.1% with dimethyl sulfoxide is used as a vehicle to apply the vehicle or the extracellular fluid containing the compound of the present invention dissolved at a concentration of interest to the cells under conditions of room temperature for 7 minutes or longer. From the obtained I_{Kr}, the absolute value of the maximum tail current based on the current value at the membrane potential where the cells have been kept is measured using analytical software (QPatch Assay software; Sophion Bioscience A/S). The maximum tail current after the application of the compound of the present invention with respect to the maximum tail current after the application of the vehicle is further calculated as the rate of inhibition to evaluate the influence of the compound of the present invention on I_{Kr}. The dilution concentration or the dilution solvent are changed as necessary.

The compounds of the present invention can be tested essentially as described above.

### Formulation Example

The compound of the present invention can be administered as a pharmaceutical composition by any conventional route, in particular enterally, for example, orally, for example, in the form of tablets or capsules, or parenterally, for example, in the form of injectable solutions or suspensions, topically, for example, in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, the oral composition can be a tablet, a granular preparation, or a capsule, each containing an excipient, a disintegrating agent, a binder, a lubricating agent, and the like, as well as an active ingredient and the like. Furthermore, the composition for injection can be prepared as a solution or a suspension, may be sterilized, and may contain a preservative, a stabilizer, a buffering agent, and the like.

### INDUSTRIAL APPLICABILITY

The compounds of the present invention have GLP-1 receptor agonist activity and are considered to be useful as a therapeutic and/or preventive agent for diseases or conditions associated with the GLP-1 receptor.

1. A compound represented by formula (I): 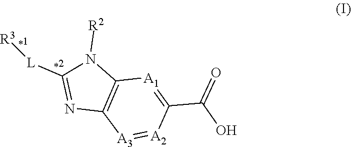
wherein
A₁ is C(R⁵) or N,
A₂ is C(R⁶) or N,
A₃ is C(R⁷) or N,
R⁵, R⁶, and R⁷ are each independently a hydrogen atom, halogen, cyano, substituted or unsubstituted alkyl, substituted or unsubstituted alkyloxy, substituted or unsubstituted aromatic heterocyclyl, or substituted or unsubstituted non-aromatic carbocyclyl,
R² is substituted or unsubstituted alkyl, or substituted or unsubstituted non-aromatic heterocyclyl,
-L- is a group represented by: 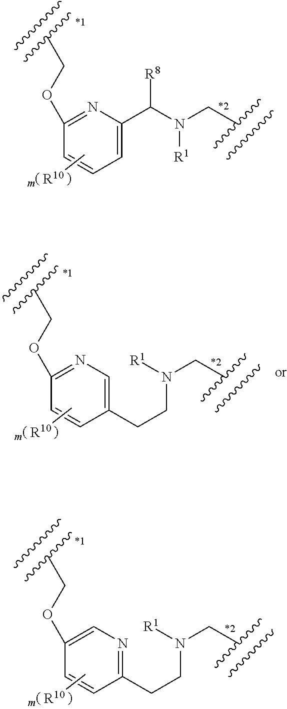
wherein
R¹ is a hydrogen atom, or substituted or unsubstituted alkyl,
R⁸ is a hydrogen atom, or substituted or unsubstituted alkyl,
R¹⁰s are each independently cyano, halogen, or substituted or unsubstituted alkyl, and
m is an integer of 1 to 3, and
R³ is phenyl optionally substituted with substituent group F, 5- or 6-membered aromatic heterocyclyl optionally substituted with substituent group F, bicyclic 9- or 10-membered aromatic heterocyclyl optionally substituted with substituent group F, or 5- to 12-membered non-aromatic heterocyclyl optionally substituted with substituent group F,
the substituent group F: halogen, cyano, alkyl, haloalkyl, alkyloxy, and haloalkyloxy,
or a pharmaceutically acceptable salt thereof.

2. The compound according to claim 1 or a pharmaceutically acceptable salt thereof, wherein R² is alkyl substituted with substituted or unsubstituted non-aromatic heterocyclyl, or alkyl substituted with substituted or unsubstituted aromatic heterocyclyl.

3. The compound according to claim 1, or a pharmaceutically acceptable salt thereof, wherein -L- is represented by: 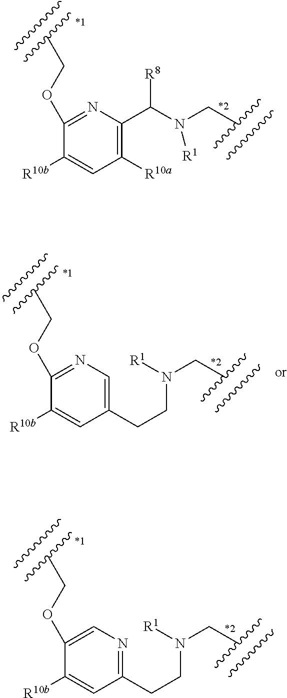
wherein R^{10a} and R^{10b} are each independently a hydrogen atom, cyano, halogen, or substituted or unsubstituted alkyl, and R¹ has the same meaning as in claim 1.

4. The compound according to claim 3 or a pharmaceutically acceptable salt thereof, wherein R^{10b} is halogen, alkyl, or haloalkyl.

5. The compound according to claim 1 or a pharmaceutically acceptable salt thereof, wherein R³ is a group below: 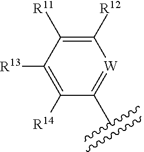
wherein
W is N, or CR¹⁵,
R¹¹ is a hydrogen atom, halogen, cyano, alkyl, haloalkyl, alkyloxy, or haloalkyloxy,
R¹² and R¹³ are each independently a hydrogen atom or halogen,
R¹⁴ and R¹⁵ are each independently a hydrogen atom, halogen, cyano, alkyl, haloalkyl, alkyloxy, or haloalkyloxy,
R¹¹ and R¹² may be taken together to form 5-membered aromatic heterocycle optionally substituted with substituent group F, or 5- to 7-membered non-aromatic heterocycle optionally substituted with substituent group F,
R¹¹ and R¹³ may be taken together to form 5-membered aromatic heterocycle optionally substituted with substituent group F, or 5- to 7-membered non-aromatic heterocycle optionally substituted with substituent group F, and
R¹³ and R¹⁴ may be taken together to form 5-membered aromatic heterocycle optionally substituted with substituent group F, or 5- to 7-membered non-aromatic heterocycle optionally substituted with substituent group F.

6. The compound according to claim 5 or a pharmaceutically acceptable salt thereof, wherein R³ is any one of groups below: 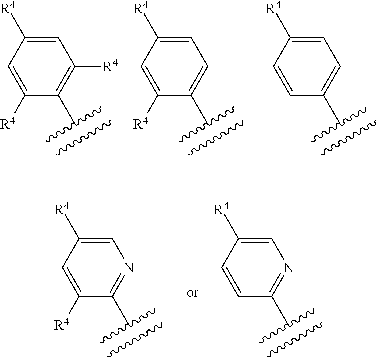
wherein R⁴s are each independently halogen, cyano, alkyl, haloalkyl, alkyloxy, or haloalkyloxy.

7. The compound according to claim 6 or a pharmaceutically acceptable salt thereof, wherein R⁴s are each independently a fluorine atom, a chlorine atom, cyano, methyl, methyloxy, or difluoromethyloxy.

8. The compound according to claim 1 or a pharmaceutically acceptable salt thereof, wherein
(i) A₁ is C(R⁵), A₂ is C(R⁶), and A₃ is C(R⁷),
(ii) A₁ is N, A₂ is C(R⁶), and A₃ is C(R⁷),
(iii) A₁ is C(R⁵), A₂ is C(R⁶), and A₃ is N, or
(iv) A₁ is N, A₂ is C(R⁶), and A₃ is N.

9. The compound according to claim 8 or a pharmaceutically acceptable salt thereof, wherein
(i) A₁ is C(R⁵), A₂ is C(R⁶), and A₃ is C(R⁷), or
(ii) A₁ is N, A₂ is C(R⁶), and A₃ is C(R⁷).

10. The compound according to claim 1 or a pharmaceutically acceptable salt thereof, wherein R⁵, R⁶, and R⁷ are each independently a hydrogen atom, halogen, alkyl, alkyloxy, or 5- or 6-membered aromatic heterocyclyl optionally substituted with a substituent group E,
wherein the substituent group E is halogen, alkyl, haloalkyl, alkyloxy, haloalkyloxy.

11. The compound according to claim 1 or a pharmaceutically acceptable salt thereof, wherein R² is oxetanylmethyl.

12. A pharmaceutical composition comprising the compound according to claim 1 or a pharmaceutically acceptable salt of thereof.

13. The pharmaceutical composition according to claim 12, which is a GLP-1 receptor agonist.
